(19) United States
(12) Patent Application Publication
(10) Pub. No.: US 2020/0038595 A1
(43) Pub. Date: Feb. 6, 2020

(54) MEDICAL SYRINGE FOR PREVENTING NEEDLE-STICK INJURY

(71) Applicant: SHANGHAI BOQIA MEDICAL DEVICE CO., LTD., SHANGHAI (CN)

(72) Inventors: YIYI ZHANG, SHANGHAI (CN); ZUREN ZHANG, SHANGHAI (CN); WEIHUA XU, SHANGHAI (CN)

(73) Assignee: SHANGHAI BOQIA MEDICAL DEVICE CO., LTD., SHANGHAI (CN)

(21) Appl. No.: 16/278,403

(22) Filed: Feb. 18, 2019

(30) Foreign Application Priority Data
Aug. 2, 2018 (CN) 201810881877.4
Aug. 2, 2018 (CN) 201810881879.3

(51) Int. Cl. A61M 5/32; A61M 5/31; A61M 5/315

(52) U.S. Cl. CPC A61M 5/321; A61M 5/3134; A61M 5/3159; A61M 5/3202; A61M 5/31576

(57) ABSTRACT

The present invention provides medical syringe for preventing needle-stick injury, wherein an inner diameter of a middle part of an elastic disengaging ring is larger than an inner diameter of a front part and an inner diameter of a rear part. The elastic disengaging ring is located between a distal wall of a syringe barrel and a needle. When the middle part of the elastic disengaging ring plugged onto the conical fitting of the syringe barrel is radially pinched flat, the proximal end of the elastically deformed elastic disengaging ring leans against the distal wall of the syringe barrel, and the front end of the elastically deformed elastic disengaging ring leans against the proximal end of the needle and pushes the needle to move forward, so that the needle is disengaged from the conical fitting of the syringe barrel.

### BACKGROUND OF THE PRESENT INVENTION

### Field of the Invention

The present invention relates to various medical syringes, specifically to various sterile syringes for single use, also specifically to various glass medical syringes, also specifically to various metal medical syringes, more specifically to various sterile syringes for single use for preventing needle-stick injury, also more specifically to various glass medical syringes for preventing needle-stick injury, also more specifically to various metal medical syringes for preventing needle-stick injury.

### Description of Related Arts

Various medical syringes can be used to inject various liquids. They can also be used to mix various liquids or mix various liquids with powders. They can also be used to flush objects. After the needle is plugged onto the conical fitting of medical syringe, various medical agents can be injected into the skin, muscle, blood vessel or other tissues and organs of the human body or animal, and various liquids can also be drawn from the tissues and organs of the human body or animal.

A conventional medical syringe includes a syringe barrel and a plunger rod. The syringe barrel has a chamber. The chamber respectively has an opening at a front end and a proximal end of the syringe barrel. A distal wall and a conical fitting are provided at a front part of the syringe barrel. The conical fitting is located at a front end of the distal wall. The syringe barrels of various sterile syringes for single use generally use distal walls with conical inclined surfaces or spherical distal walls. The syringe barrels of various glass medical syringes and metal medical syringes generally use plane distal walls. The conical fitting may be Luer slip centric or Luer slip excentric. The conical fitting has a passageway. The passageway joins the chamber through. A needle may be mounted on the conical fitting of the syringe barrel. A peripheral flange is provided at a rear part of the syringe barrel. The syringe barrel may be sufficiently transparent. A volume scale may be provided at an outer surface of the syringe barrel. A thumbpress is provided at a proximal end of the plunger rod and a stopper is provided at a front part of the plunger rod. After the plunger rod is plugged into the chamber from the opening in the proximal end of the chamber, the plunger rod is capable of moving forwards and backwards in the chamber by manipulating the thumbpress of the plunger rod, and an outer wall of the stopper and an inner wall of the chamber form a sealing fit of the medical syringe.

The unsatisfactory aspect of the conventional medical syringe is that after the medical syringe is used, the contaminated needle exposed is very easy to cause damage to the human body, and because the needle is tightly engaged with the conical fitting of the syringe barrel of the syringe, the actions of removing the contaminated needle and putting back the sleeve to close the needle head of the needle are the mostly highly dangerous actions that cause needle-stick injuries of health workers. A 2014 study sponsored by WHO, which focused on the most recent available data, estimated that in 2010, up to 1.7 million people were infected with hepatitis B virus, up to 315 000 with hepatitis C virus and as many as 33 800 with HIV due to unsafe injection, therefore, WHO is urging countries to transition, by 2020, to the exclusive use of the new “smart” syringes. The new injection safety guidelines and policies issued by WHO on Feb. 23, 2015 provide detailed recommendations suggesting that syringes should be engineered with safety device to prevent health workers from infections resulted by “needle-stick” injuries.

For a medical syringe for preventing needle-stick injury described in US Patent US20060052748A1, a needle shield hinged to the syringe barrel is provided at the front part of the syringe barrel of the medical syringe. When in use, the needle shield is pivoted from a closed needle protecting position to an open position. After use, the needle shield is pivoted from an open position to a closed needle protecting position, so as to prevent the contaminated needle from injuring the human body. For a medical syringe for preventing needle-stick injury described in US Patents US20150182704A1 and US20160367762A1, the medical syringe is provided with a needle sheath that is capable of moving forwards and backwards at the periphery of the syringe barrel. When in use, the needle sheath is moved backwards at the periphery of the syringe barrel to reveal the needle. After use, the needle sheath is moved forwards to the needle at the periphery of the syringe barrel, and the needle is covered, so as to prevent the contaminated needle from injuring the human body. The first unsatisfactory aspect of the above-mentioned two medical syringes for preventing needle-stick injury is that the medical syringe for preventing needle-stick injury have more parts, and is more complicated and more difficult to assemble than conventional medical syringes, therefore the cost is higher. According to a WHO survey, the cost of a medical syringe for preventing needle-stick injury with a needle cover is more than twice the cost of a conventional medical syringe. The second unsatisfactory aspect of the above-mentioned two medical syringes for preventing needle-stick injury is that the needle cover increases injection operation steps, and the needle cover on the syringe barrel affects the injection operation of the medical syringe for preventing needle-stick injury, which is easy to cause operational errors. The third unsatisfactory aspect of the above-mentioned two types of medical syringes for preventing needle-stick injury is that the plastic medical syringe is fixed with the metal needle, needle cannot be replaced with needles of other specifications before use. The overall volume after use is relatively large, which brings troubles to the subsequent destruction treatment, and the plastic material and the metal material cannot be classified and recycled. The fourth unsatisfactory aspect of the above-mentioned two medical syringes for preventing needle-stick injury is that the cost for installing the needle cover on various glass medical syringes and metal medical syringes is higher, and it is difficult to achieve sterilization requirements before use.

It can be clearly seen from the above that the existing medical syringe needs to be further improved, such that the production cost of the medical syringe is lower, the medical syringe is safer and more convenient to use, and the medical syringe is more easily recycled or destroyed after use.

### SUMMARY OF THE PRESENT INVENTION

The first purpose of the present invention is to provide a medical syringe for preventing needle-stick injury, which adopts a technical feature that an inner diameter of a middle part of an elastic disengaging ring is larger than an inner diameter of a front part and an inner diameter of a rear part, and adopts a technical solution that the elastic disengaging ring is located between a distal wall of a syringe barrel and a needle. When the middle part of the elastic disengaging ring plugged onto the conical fitting of the syringe barrel is radially pinched flat, the proximal end of the elastically deformed elastic disengaging ring leans against the distal wall of the syringe barrel, the front end of the elastically deformed elastic disengaging ring leans against the proximal end of the needle and pushes the needle to move forward, so that the needle is disengaged from the conical fitting of the syringe barrel. The technical effect that the medical syringe for preventing needle-stick injury will not touch the needle stick during operation is achieved, so as to prevent the contaminated needle from injuring the human body. Since the inner diameter of the middle part of the elastic disengaging ring is larger than the inner diameter of the front part and the inner diameter of the rear part, only a small force is required to compress the front part and the rear part of the elastic disengaging ring to each other without affecting the operation to plug the needle onto the conical fitting of the syringe barrel. When the middle part of the elastic disengaging ring is radially pinched flat, the middle part of the elastic disengaging ring is squeezed between the front part and the rear part thereof, so that the axial length of the elastic disengaging ring is elongated and the rigidity is increased, which facilitates the needle being pushed forward and then disengaging from the conical fitting of the syringe barrel. The elastic disengaging ring fitted over the conical fitting of the syringe barrel does not affect the injection operation of the medical syringe for preventing needle-stick injury, or increase the injection procedures.

The second purpose of the present invention is to provide a medical syringe for preventing needle-stick injury, which adopts a technical feature an inner diameter of a rear part of the elastic disengaging ring is larger than an inner diameter of a front part of the elastic disengaging ring and an outer diameter of the conical fitting of the syringe barrel, and adopts a technical solution that the elastic disengaging ring is located between the distal wall of the syringe barrel and the needle. When the rear part of the elastic disengaging ring on the conical fitting of the syringe barrel is radially pinched flat, the proximal end of the elastically deformed elastic disengaging ring leans against the distal wall of the syringe barrel and moves along the distal wall, the front end of the elastically deformed elastic disengaging ring leans against the proximal end of the needle and pushes the needle to move forward, so that the needle is disengaged from the conical fitting of the syringe barrel. The technical effect that the medical syringe for preventing needle-stick injury will not touch the needle stick during operation is achieved, so as to prevent the contaminated needle from injuring the human body. The elastic disengaging ring fitted over the conical fitting of the syringe barrel does not affect the injection operation of the medical syringe for preventing needle-stick injury, or increase the injection procedures.

The third purpose of the present invention is to provide a medical syringe for preventing needle-stick injury, which has only one more elastic disengaging ring over the conical fitting of the syringe barrel than the conventional medical syringe. The cost of the elastic disengaging ring is less than 3% of the cost of the medical syringe for preventing needle-stick injury, and the assembly is also convenient. The elastic disengaging ring can be used to form a sterile syringe for single use for preventing needle-stick injury by directly putting the elastic disengaging ring on the conical fitting of the syringe barrel of conventional various sterile syringes for single use. For sterile syringe for single use for preventing needle-stick injury in the present invention, needle can be replaced with needles of other specifications before use; after use, the contaminated metal needles and plastic medical syringes can be classified into a collection box, the plastic material and the metal material can be classified and recycled, or subsequently destroyed. Conventional various glass medical syringes and metal medical syringes can be routinely cleaned and sterilized before use. When in use, the elastic disengaging ring can be put on the conical fitting of the syringe barrel to form a glass medical syringe for preventing needle-stick injury or a metal medical syringe for preventing needle-stick injury, the elastic disengaging ring and the needle are destroyed after use. Since the medical syringe for preventing needle-stick injury of the present invention can directly use conventional syringe barrels, plunger rods and needles of conventional various sterile syringes for single use, various glass medical syringes or various metal medical syringes, therefore existing processing, assembly and packaging techniques and equipment can be directly applied, thereby reducing the product cost. The conventional needles which can be used both for various medical syringes with an outer conical locking fitting and for various medical syringes without the outer conical locking fitting will not be affected.

The purposes of the present invention are implemented through the following technical solutions.

The medical syringe for preventing needle-stick injury of the present invention includes a syringe barrel and a plunger rod. A distal wall and a conical fitting are provided at a front part of the syringe barrel, the conical fitting is located at a front end of the distal wall. The syringe barrels of various sterile syringes for single use generally use distal walls of conical inclined surfaces or spherical distal walls. The syringe barrels of various glass medical syringes and metal medical syringes generally use plane distal walls. The syringe barrel has a chamber. The chamber respectively has an opening at a front end and a proximal end of the syringe barrel. The conical fitting of the syringe barrel may be Luer slip centric or Luer slip excentric. The conical fitting has a passageway. The passageway joins the chamber through. A peripheral flange is provided at a rear part of the syringe barrel. A volume scale may be provided at an outer surface of the syringe barrel.

A thumbpress is provided at a proximal end of the plunger rod, and a stopper is provided at a front part of the plunger rod. After the plunger rod is plugged into the chamber from the opening in the proximal end of the chamber, the plunger rod is capable of moving forwards and backwards in the chamber by manipulating the thumbpress of the plunger rod. The stopper and the plunger rod may be an integral type or a partable type. An outer wall of the stopper and an inner wall of the chamber form a sealing fit of the medical syringe.

In the first solution of the medical syringe for preventing needle-stick injury of the present invention, a needle and an elastic disengaging ring may be plugged onto the conical fitting of the syringe barrel. The elastic disengaging ring is located between the distal wall of the syringe barrel and the needle. An inner diameter of a middle part of the elastic disengaging ring is larger than an inner diameter of a front part and an inner diameter of a rear part. When the middle part of the elastic disengaging ring on the conical fitting of the syringe barrel is radially pinched flat, the proximal end of the elastically deformed elastic disengaging ring leans against the distal wall of the syringe barrel, and the front end of the elastically deformed elastic disengaging ring leans against the proximal end of the needle to push the needle to move forward, so that the needle is disengaged from the conical fitting of the syringe barrel.

In the first solution of the medical syringe for preventing needle-stick injury of the present invention, the elastic disengaging ring is capable of moving forwards and backwards on the conical fitting of the syringe barrel. When the needle and the elastic disengaging ring are plugged onto the conical fitting of the syringe barrel, the needle is connected to the conical fitting of the syringe barrel, the elastic disengaging ring is located between the distal wall of the syringe barrel and the needle. When the middle part of the elastic disengaging ring on the conical fitting of the syringe barrel is radially pinched flat, a proximal end of the elastically deformed elastic disengaging ring leans against the distal wall of the syringe barrel, and a front end of the elastically deformed elastic disengaging ring leans against a proximal end of the needle to push the needle to move forward, so that the needle is disengaged from the conical fitting of the syringe barrel.

The inner diameter of the front part of the elastic disengaging ring may be smaller than an outer diameter of the conical fitting of the syringe barrel. When the inner diameter of the front part of the elastic disengaging ring is elastically deformed and plugged onto the conical fitting of the syringe barrel, the elastic disengaging ring is constrained on the conical fitting of the syringe barrel, and the elastic disengaging ring moves forwards and backwards on the conical fitting of the syringe barrel under an external force.

The inner diameter of the rear part of the elastic disengaging ring may be larger than the outer diameter of the conical fitting of the syringe barrel. A conical inclined surface may be provided on the distal wall of the syringe barrel. When the middle part of the elastic disengaging ring on the conical fitting of the syringe barrel is radially pinched flat, the proximal end of the elastically deformed elastic disengaging ring leans against the distal wall of the syringe barrel and moves along the conical inclined surface on the distal wall, and the front end of the elastically deformed elastic disengaging ring leans against the proximal end of the needle and pushes the needle to move forward, so that the needle is disengaged from the conical fitting of the syringe barrel.

In the first solution of the medical syringe for preventing needle-stick injury of the present invention, the rear part of the elastic disengaging ring may be fixedly mounted at the front part of the distal wall of the syringe barrel through snap-fitting, bonding, welding or other means. When the needle is plugged onto the conical fitting of the syringe barrel, the needle is connected to the conical fitting of the syringe barrel, and the elastic disengaging ring is located between the distal wall of the syringe barrel and the needle. When the middle part of the elastic disengaging ring on the conical fitting of the syringe barrel is radially pinched flat, a front end of the elastically deformed elastic disengaging ring leans against a proximal end of the needle and pushes the needle to move forward, so that the needle is disengaged from the conical fitting of the syringe barrel.

In the first solution of the medical syringe for preventing needle-stick injury of the present invention, the front part of the elastic disengaging ring may be fixedly mounted at the rear part of the needle through snap-fitting, bonding, welding or other means. When the needle is plugged onto the conical fitting of the syringe barrel, the needle is connected to the conical fitting of the syringe barrel, and the elastic disengaging ring is located between the distal wall of the syringe barrel and the needle. When the middle part of the elastic disengaging ring mounted on the rear part of the needle is radially pinched flat, a proximal end of the elastically deformed elastic disengaging ring leans against the distal wall of the syringe barrel, and a front end of the elastically deformed elastic disengaging ring leans against a proximal end of the needle and pushes the needle to move forward, so that the needle is disengaged from the conical fitting of the syringe barrel.

In the second solution of the medical syringe for preventing needle-stick injury of the present invention, a needle and an elastic disengaging ring are capable of being plugged onto the conical fitting of the syringe barrel. The elastic disengaging ring is located between the distal wall of the syringe barrel and the needle. An inner diameter of a rear part of the elastic disengaging ring is larger than an inner diameter of the front part of the elastic disengaging ring and an outer diameter of the conical fitting of the syringe barrel. The elastic disengaging ring is capable of moving forwards and backwards on the conical fitting of the syringe barrel. When the rear part of the elastic disengaging ring on the conical fitting of the syringe barrel is radially pinched flat, the proximal end of the elastically deformed elastic disengaging ring leans against the distal wall of the syringe barrel and moves along the distal wall, and the front end of the elastically deformed elastic disengaging ring leans against the proximal end of the needle to push the needle to move forward, so that the needle is disengaged from the conical fitting of the syringe barrel.

In the second solution of the medical syringe for preventing needle-stick injury of the present invention, an inner diameter of a front part of the elastic disengaging ring may be smaller than an outer diameter of the conical fitting of the syringe barrel. When the inner diameter of the front part of the elastic disengaging ring is elastically deformed and plugged onto the conical fitting of the syringe barrel, the elastic disengaging ring is constrained on the conical fitting of the syringe barrel, and the elastic disengaging ring moves forwards and backwards on the conical fitting of the syringe barrel under an external force.

In the second solution of the medical syringe for preventing needle-stick injury of the present invention, a conical inclined surface may be provided on the distal wall of the syringe barrel. When the rear part of the elastic disengaging ring on the conical fitting of the syringe barrel is radially pinched flat, the proximal end of the elastically deformed elastic disengaging ring leans against the distal wall of the syringe barrel and moves along the conical inclined surface, and the front end of the elastically deformed elastic disengaging ring leans against the proximal end of the needle and pushes the needle to move forward, so that the needle is disengaged from the conical fitting of the syringe barrel.

In the third solution of the medical syringe for preventing needle-stick injury of the present invention, a needle and an elastic disengaging ring are plugged onto the conical fitting of the syringe barrel. A front part of the elastic disengaging ring is fixedly snap-fitted on a locking fitting of a rear part of the needle by a bayonet. An inner diameter of a rear part of the elastic disengaging ring is larger than an inner diameter of the front part of the elastic disengaging ring and an outer diameter of the conical fitting of the syringe barrel. When the needle is plugged onto the conical fitting of the syringe barrel, the needle is connected to the conical fitting of the syringe barrel, and the elastic disengaging ring is located between the distal wall of the syringe barrel and the needle. When the rear part of the elastic disengaging ring mounted on the rear part of the needle is radially pinched flat, a proximal end of the elastically deformed elastic disengaging ring leans against the distal wall of the syringe barrel, and a front end of the elastically deformed elastic disengaging ring leans against a proximal end of the needle and pushes the needle to move forward, so that the needle is disengaged from the conical fitting of the syringe barrel.

In the third solution of the medical syringe for preventing needle-stick injury of the present invention, a conical inclined surface may be provided on the distal wall of the syringe barrel. When the rear part of the elastic disengaging ring on the conical fitting of the syringe barrel is radially pinched flat, the proximal end of the elastically deformed elastic disengaging ring leans against the distal wall of the syringe barrel and moves along the conical inclined surface, and the front end of the elastically deformed elastic disengaging ring leans against the proximal end of the needle and pushes the needle to move forward, so that the needle is disengaged from the conical fitting of the syringe barrel.

### BRIEF DESCRIPTION OF THE DRAWINGS

FIG. 1 is a perspective view of a medical syringe for preventing needle-stick injury according to embodiment 1 of the present invention;

FIG. 2 is a perspective view of the medical syringe for preventing needle-stick injury in FIG. 1 after the middle part of the elastic disengaging ring is radially pinched flat and the needle is moved forward;

FIG. 3 is a front view of the medical syringe for preventing needle-stick injury in FIG. 1;

FIG. 4 is a cross-sectional view taken along line 4-4 in FIG. 3;

FIG. 5 is a front view of the medical syringe for preventing needle-stick injury in FIG. 2;

FIG. 6 is a cross-sectional view taken along line 6-6 in FIG. 5;

FIG. 7 is a perspective view of a syringe barrel and a plunger rod in FIG. 1;

FIG. 8 is a perspective view of the needle in FIG. 1;

FIG. 9 is a perspective view of the elastic disengaging ring in FIG. 1;

FIG. 10 is a perspective view after the middle part of the elastic disengaging ring in FIG. 2 is radially pinched flat;

FIG. 11 is a perspective view when the elastic disengaging ring of the medical syringe for preventing needle-stick injury in FIG. 1 is fixedly mounted at the front part of the distal wall of the syringe barrel through bonding or welding;

FIG. 12 is a perspective view of the medical syringe for preventing needle-stick injury in a state that the elastic disengaging ring is fixedly mounted at the front part of the distal wall of the syringe barrel through bonding or welding in FIG. 11;

FIG. 13 is a perspective view when the elastic disengaging ring of the medical syringe for preventing needle-stick injury in FIG. 1 is fixedly mounted at the locking fitting at the proximal end of the needle through snap-fitting;

FIG. 14 is a perspective view when the elastic disengaging ring of in FIG. 13 is fixedly mounted at the locking fitting at the proximal end of the needle through snap-fitting;

FIG. 15 is a perspective view of the elastic disengaging ring in FIG. 14;

FIG. 16 is a perspective view of the medical syringe for preventing needle-stick injury according to embodiment 2 of the present invention;

FIG. 17 is a perspective view of the medical syringe for preventing needle-stick injury in FIG. 16 after the middle part of the elastic disengaging ring is radially pinched flat and the needle is moved forward;

FIG. 18 is a front view of the medical syringe for preventing needle-stick injury in FIG. 16;

FIG. 19 is a cross-sectional view taken along line 19-19 in FIG. 18;

FIG. 20 is a front view of the medical syringe for preventing needle-stick injury in FIG. 17;

FIG. 21 is a cross-sectional view taken along line 21-21 in FIG. 20;

FIG. 22 is a perspective view of the syringe barrel and the plunger rod in FIG. 16;

FIG. 23 is a perspective view of the needle in FIG. 16;

FIG. 24 is a perspective view of the elastic disengaging ring in FIG. 16;

FIG. 25 is a perspective view after the middle part of the elastic disengaging ring in FIG. 17 is radially pinched flat;

FIG. 26 is perspective view of the medical syringe for preventing needle-stick injury according to embodiment 3 of the present invention;

FIG. 27 is a perspective view of the medical syringe for preventing needle-stick injury in FIG. 26 after the middle part of the elastic disengaging ring is radially pinched flat and the needle is moved forward;

FIG. 28 is a front view of the medical syringe for preventing needle-stick injury in FIG. 26;

FIG. 29 is a cross-sectional view taken along line 29-29 in FIG. 28;

FIG. 30 is a front view of the medical syringe for preventing needle-stick injury in FIG. 27;

FIG. 31 is a cross-sectional view taken along line 31-31 in FIG. 30;

FIG. 32 is a perspective view of the syringe barrel and the plunger rod in FIG. 26;

FIG. 33 is a perspective view of the needle in FIG. 26;

FIG. 34 is a perspective view of the elastic disengaging ring in FIG. 26;

FIG. 35 is a perspective view after the middle part of the elastic disengaging ring in FIG. 27 is radially pinched flat;

FIG. 36 is perspective view of the medical syringe for preventing needle-stick injury according to embodiment 4 of the present invention;

FIG. 37 is a perspective view of the medical syringe for preventing needle-stick injury in FIG. 36 after the middle part of the elastic disengaging ring is radially pinched flat and the needle is moved forward;

FIG. 38 is a front view of the medical syringe for preventing needle-stick injury in FIG. 36;

FIG. 39 is a cross-sectional view taken along line 39-39 in FIG. 38;

FIG. 40 is a front view of the medical syringe for preventing needle-stick injury in FIG. 37;

FIG. 41 is a cross-sectional view taken along line 41-41 in FIG. 40;

FIG. 42 is a perspective view of the syringe barrel and the plunger rod in FIG. 36;

FIG. 43 is a perspective view of the needle in FIG. 36;

FIG. 44 is a perspective view of the elastic disengaging ring in FIG. 36;

FIG. 45 is a perspective view after the rear part of the elastic disengaging ring in FIG. 37 is radially pinched flat;

FIG. 46 is perspective view of the medical syringe for preventing needle-stick injury according to embodiment 5 of the present invention;

FIG. 47 is a perspective view of the medical syringe for preventing needle-stick injury in FIG. 46 after the middle part of the elastic disengaging ring is radially pinched flat and the needle is moved forward;

FIG. 48 is a front view of the medical syringe for preventing needle-stick injury in FIG. 46;

FIG. 49 is a cross-sectional view taken along line 49-49 in FIG. 48;

FIG. 50 is a front view of the medical syringe for preventing needle-stick injury in FIG. 47;

FIG. 51 is a cross-sectional view taken along line 51-51 in FIG. 50;

FIG. 52 is a perspective view of the syringe barrel and the plunger rod in FIG. 46;

FIG. 53 is a perspective view of the needle in FIG. 46;

FIG. 54 is a perspective view of the elastic disengaging ring in FIG. 46;

FIG. 55 is a perspective view when the rear part of the elastic disengaging ring in FIG. 47 is radially pinched flat;

FIG. 56 is perspective view of the medical syringe for preventing needle-stick injury according to embodiment 6 of the present invention;

FIG. 57 is a perspective view of the medical syringe for preventing needle-stick injury in FIG. 56 after the rear part of the elastic disengaging ring is radially pinched flat and the needle is moved forward;

FIG. 58 is a perspective view when the elastic disengaging ring in FIG. 56 is fixedly mounted at the locking fitting at the proximal end of the needle through snap-fitting;

FIG. 59 is a perspective view of the elastic disengaging ring in FIG. 58;

FIG. 60 is a perspective view when the rear part of the elastic disengaging ring fixedly mounted at the locking fitting at the proximal end of the needle through snap-fitting in FIG. 57 is radially pinched flat;

FIG. 61 is a perspective view after the rear part of the elastic disengaging ring in FIG. 60 is radially pinched flat;

FIG. 62 is perspective view of the medical syringe for preventing needle-stick injury according to embodiment 7 of the present invention;

FIG. 63 is a perspective view of the medical syringe for preventing needle-stick injury in FIG. 62 after the rear part of the elastic disengaging ring is radially pinched flat and the needle is moved forward;

FIG. 64 is a front view of the medical syringe for preventing needle-stick injury in FIG. 62;

FIG. 65 is a cross-sectional view taken along line 65-65 in FIG. 64;

FIG. 66 is a front view of the medical syringe for preventing needle-stick injury in FIG. 63;

FIG. 67 is a cross-sectional view taken along line 67-67 in FIG. 66;

FIG. 68 is a perspective view of the syringe barrel and the plunger rod in FIG. 62;

FIG. 69 is a perspective view of the needle in FIG. 62;

FIG. 70 is a perspective view of the elastic disengaging ring in FIG. 62;

FIG. 71 is a perspective view after the rear part of the elastic disengaging ring in FIG. 63 is radially pinched flat.

### DETAILED DESCRIPTION OF THE PREFERRED EMBODIMENTS

Exemplary embodiments of the medical syringe for preventing needle-stick injury of the present invention are described using examples and with reference to the accompanying drawings. The scope of the present invention is subject to the claims. It should be noted that some or all of the accompanying drawings are simple drawings merely provided for illustrating the exemplary embodiments of the present invention. Therefore, actual sizes of the components are not show in the drawings. Practical methods for achieving the above and other objectives and advantages of the present invention can be better comprehended through detailed description of the exemplary embodiments. In the accompanying drawings and following description, the terms “proximal” and “rear” refer to a position close to a manipulator of the medical syringe, and the term “front” and “distal” refers to a position away from a manipulator of the medical syringe. Other terms related to positions and directions may be further understood according to the accompanying drawings and the following description.

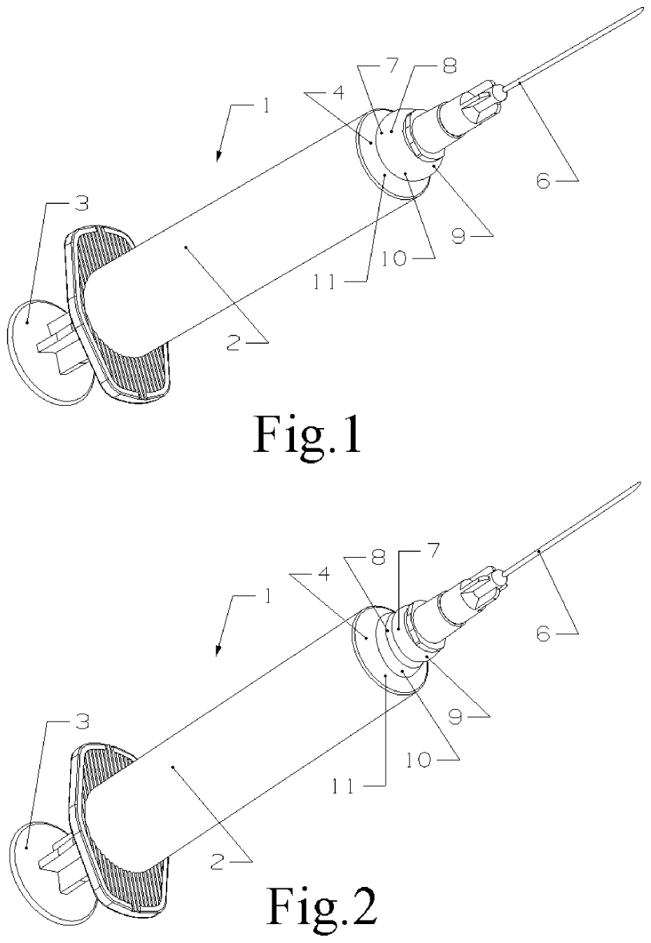

FIGS. 1-10 show a medical syringe 1 for preventing needle-stick injury according to a first embodiment of the present invention.

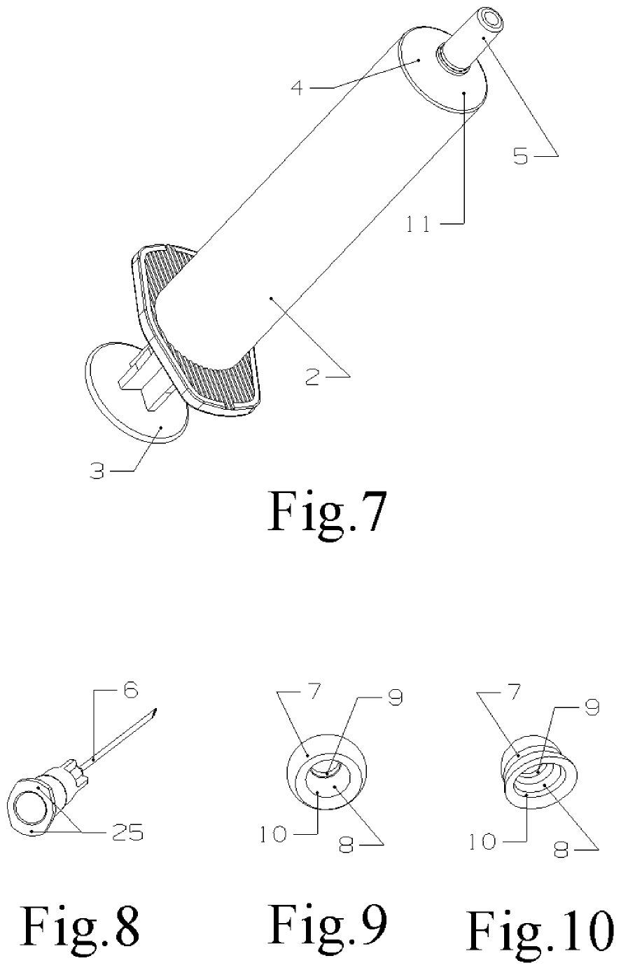

As shown in FIG. 1 and FIG. 7, the medical syringe 1 for preventing needle-stick injury includes a syringe barrel 2 and a plunger rod 3. A distal wall 4 and a conical fitting 5 which is Luer slip centric are provided at a front part of the syringe barrel 2, and the conical fitting 5 is located at a front end of the distal wall 4. After the plunger rod 3 is plugged into the syringe barrel 2 from an opening in a proximal end of the syringe barrel 2, the plunger rod 3 is capable of moving forwards and backwards in the syringe barrel 2. The medical syringe 1 for preventing needle-stick injury may be a sterile syringe for single use for preventing needle-stick injury of middle volume.

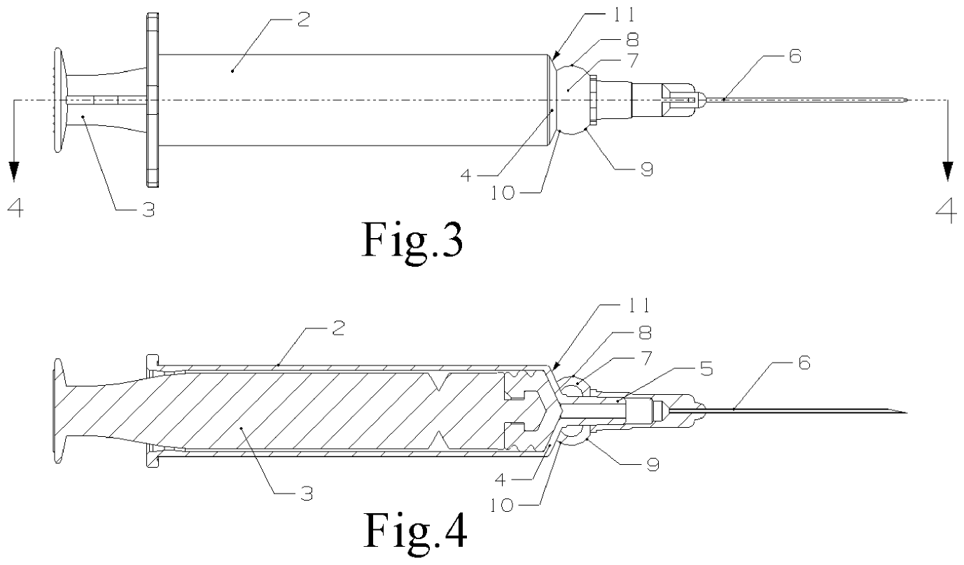

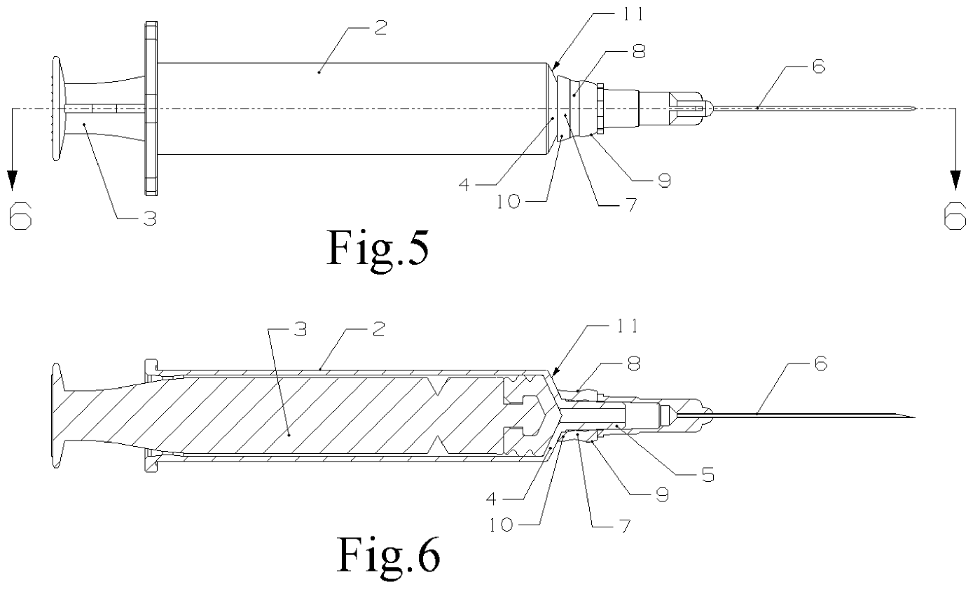

A needle 6 and an elastic disengaging ring 7 may be plugged onto the conical fitting 5 of the syringe barrel 2. The elastic disengaging ring 7 is located between the distal wall 4 of the syringe barrel 2 and the needle 6 (refer to FIG. 1). An inner diameter of a middle part 8 of the elastic disengaging ring 7 is larger than an inner diameter of a front part 9 and an inner diameter of a rear part 10 (refer to FIG. 9). The elastic disengaging ring 7 is capable of moving forwards and backwards on the conical fitting 5 of the syringe barrel 2. When the needle 6 and the elastic disengaging ring 7 are plugged onto the conical fitting 5 of the syringe barrel 2, the needle 6 is connected to the conical fitting 5 of the syringe barrel 2, and the elastic disengaging ring 7 is located between the distal wall 4 of the syringe barrel 2 and the needle 6 (refer to FIG. 1, FIG. 3 and FIG. 4). When the middle part of the elastic disengaging ring 7 on the conical fitting 5 of the syringe barrel 2 is radially pinched flat, a proximal end of the elastically deformed elastic disengaging ring 7 leans against the distal wall 4 of the syringe barrel 2, and a front end of the elastically deformed elastic disengaging ring 7 leans against a proximal end of the needle 6 and pushes the needle 6 to move forward, so that the needle 6 is disengaged from the conical fitting 5 of the syringe barrel 2 (refer to FIG. 2, FIG. 5, FIG. 6 and FIG. 10).

The inner diameter of the front part 9 of the elastic disengaging ring 7 is smaller than an outer diameter of the conical fitting 5 of the syringe barrel 2. When the inner diameter of the front part 9 of the elastic disengaging ring 7 is plugged onto the conical fitting 5 of the syringe barrel 2 after being elastically deformed, and the elastic disengaging ring 7 is constrained on the conical fitting 5 of the syringe barrel 2, the elastic disengaging ring 7 moves forwards and backwards on the conical fitting 5 of the syringe barrel 2 only under an external force (refer to FIG. 1, FIG. 3, FIG. 4 and FIG. 9).

The inner diameter of the rear part 10 of the elastic disengaging ring 7 is larger than the outer diameter of the conical fitting 5 of the syringe barrel 2. A conical inclined surface 11 is provided on the distal wall 4 of the syringe barrel 2 (refer to FIG. 7). When the middle part 8 of the elastic disengaging ring 7 on the conical fitting 5 of the syringe barrel 2 is pinched flat in a radial direction, the proximal end of the elastically deformable elastic disengaging ring 7 leans against the distal wall 4 of the syringe barrel 2 and moves along the conical inclined surface 11 on the distal wall 4, and the front end of the elastically deformed elastic disengaging ring 7 leans against the proximal end of the needle 6 and pushes the needle 6 to move forward, so that the needle 6 is disengaged from the conical fitting 5 of the syringe barrel 2 (refer to FIG. 2, FIG. 5, FIG. 6 and FIG. 10).

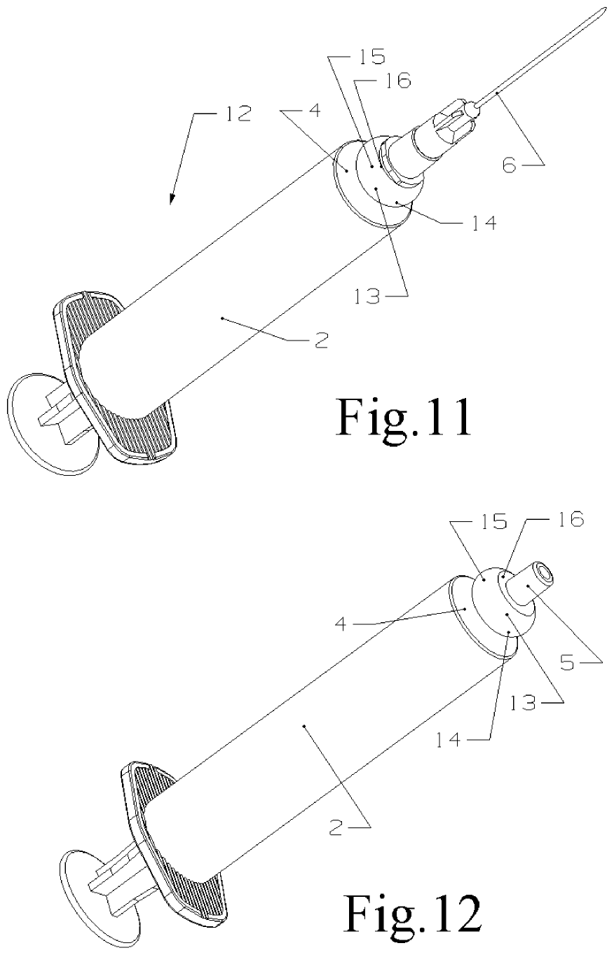

FIG. 11 and FIG. 12 show a medical syringe 12 for preventing needle-stick injury of the present invention, wherein a rear part 14 of an elastic disengaging ring 13 is fixedly mounted at a front end of the distal wall 4 of the syringe barrel 2 through bonding or welding. When the needle 6 is plugged onto the conical fitting 5 of the syringe barrel 2, the needle 6 is connected to the conical fitting 5 of the syringe barrel 2, and the elastic disengaging ring 13 is located between the distal wall 4 of the syringe barrel 2 and the needle 6. When a middle part 15 of the elastic disengaging ring 13 on the conical fitting 5 of the syringe barrel 2 is radially pinched flat, a front end 16 of the elastically deformed elastic disengaging ring 13 leans against the proximal end of the needle 6, and pushes the needle 6 to move forward, so that the needle 6 is disengaged from the conical fitting 5 of the syringe barrel 2.

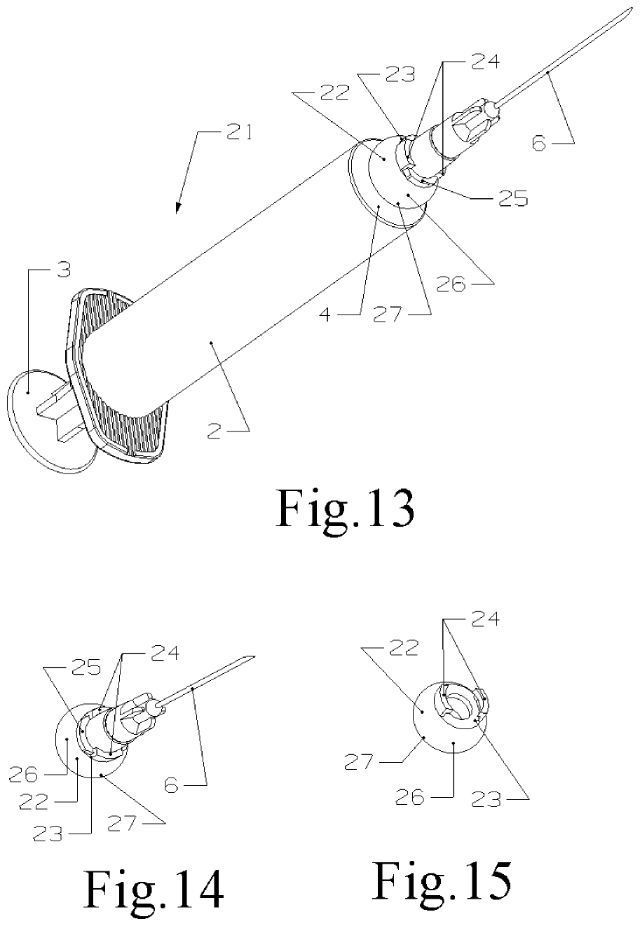

FIG. 13 to FIG. 15 show a medical syringe 21 for preventing needle-stick injury of the present invention, wherein a front part 23 of an elastic disengaging ring 22 is fixedly mounted on a locking fitting 25 (refer to FIG. 7) at the rear part of the needle 6 through snap-fitting. When the needle 6 is plugged onto the conical fitting 5 of the syringe barrel 2, the needle 6 is connected to the conical fitting 5 of the syringe barrel 2, and an elastic disengaging ring 22 is located between the distal wall 4 of the syringe barrel 2 and the needle 6. When a middle part 26 of the elastic disengaging ring 22 mounted on the rear part of the needle 6 is radially pinched flat, a rear part 27 of the elastically deformed elastic disengaging ring 22 leans against the distal wall 4 of the syringe barrel 2, and a front part 23 of the elastically deformed elastic disengaging ring 22 leans against the proximal end of the needle 6 and pushes the needle 6 to move forward, so that the needle 6 is disengaged from the conical fitting 5 of the syringe barrel 2.

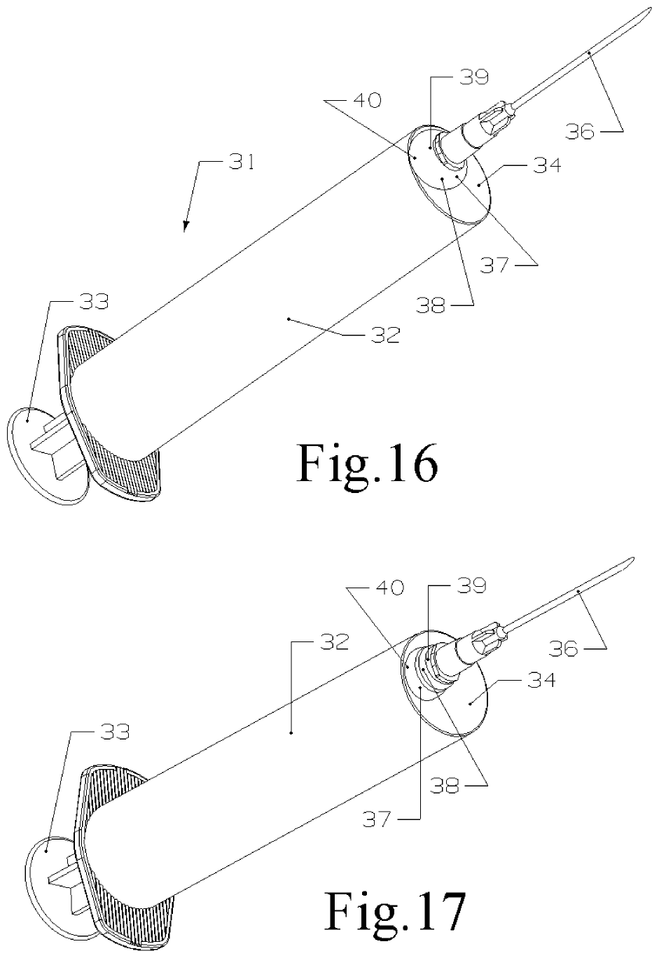

FIGS. 16-25 show a medical syringe 31 for preventing needle-stick injury according to a second embodiment of the present invention.

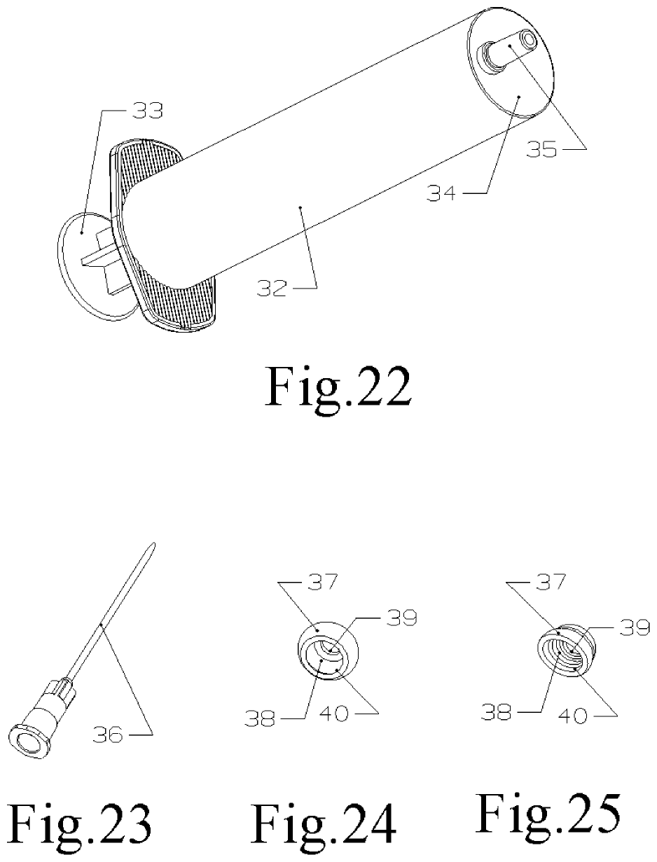

As shown in FIG. 16 and FIG. 22, the medical syringe 31 for preventing needle-stick injury includes a syringe barrel 32 and a plunger rod 33. A distal wall 34 and a conical fitting 35 that is Luer slip excentric are provided at a front part of the syringe barrel 32. The distal wall 34 of the syringe barrel 32 is spherical. The conical fitting 35 is located at a front end of the distal wall 34. After the plunger rod 33 is plugged into the syringe barrel 32 from an opening in a proximal end of the syringe barrel 32, the plunger rod 33 is capable of moving forwards and backwards in the syringe barrel 32. The medical syringe 31 for preventing needle-stick injury may be a sterile syringe for single use for preventing needle-stick injury which has a large volume and large diameter of syringe barrel.

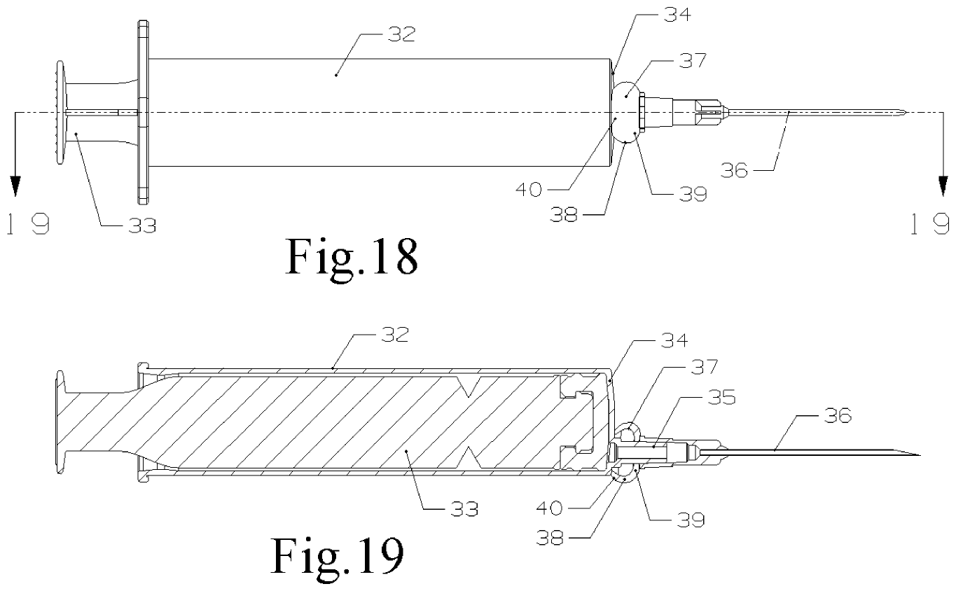

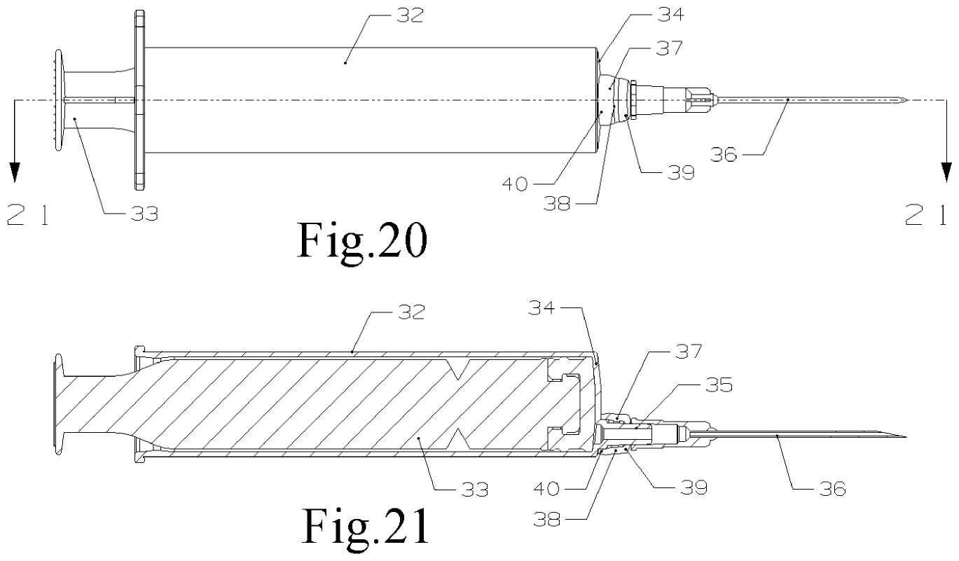

A needle 36 and an elastic disengaging ring 37 may be plugged onto the conical fitting 35 of the syringe barrel 32. The elastic disengaging ring 37 is located between the distal wall 34 of the syringe barrel 32 and the needle 36 (refer to FIG. 16). An inner diameter of a middle part 38 of the elastic disengaging ring 37 is larger than an inner diameter of a front part 39 and an inner diameter of a rear part 40 (refer to FIG. 24). The elastic disengaging ring 37 is capable of moving forwards and backwards on the conical fitting 35 of the syringe barrel 32. When the needle 36 and the elastic disengaging ring 37 are plugged onto the conical fitting 35 of the syringe barrel 32, the needle 36 is connected to the conical fitting 35 of the syringe barrel 32, and the elastic disengaging ring 37 is located between the distal wall 34 of the syringe barrel 32 and the needle 36 (refer to FIG. 16, FIG. 18 and FIG. 19). When the middle part of the elastic disengaging ring 37 on the conical fitting 35 of the syringe barrel 32 is radially pinched flat, a proximal end of the elastically deformed elastic disengaging ring 37 leans against the distal wall 34 of the syringe barrel 32, and a front end of the elastically deformed elastic disengaging ring 37 leans against a proximal end of the needle 36 and pushes the needle 36 to move forward, so that the needle 36 is disengaged from the conical fitting 35 of the syringe barrel 32 (refer to FIG. 17, FIG. 20, FIG. 21 and FIG. 25).

The inner diameter of the front part 39 of the elastic disengaging ring 37 is smaller than an outer diameter of the conical fitting 35 of the syringe barrel 32. When the inner diameter of the front part 39 of the elastic disengaging ring 37 is elastically deformed and plugged onto the conical fitting 35 of the syringe barrel 32, the elastic disengaging ring 37 is constrained on the conical fitting 35 of the syringe barrel 32, the elastic disengaging ring 37 moves forwards and backwards on the conical fitting 35 of the syringe barrel 32 only under an external force (refer to FIG. 16, FIG. 18, FIG. 19 and FIG. 24).

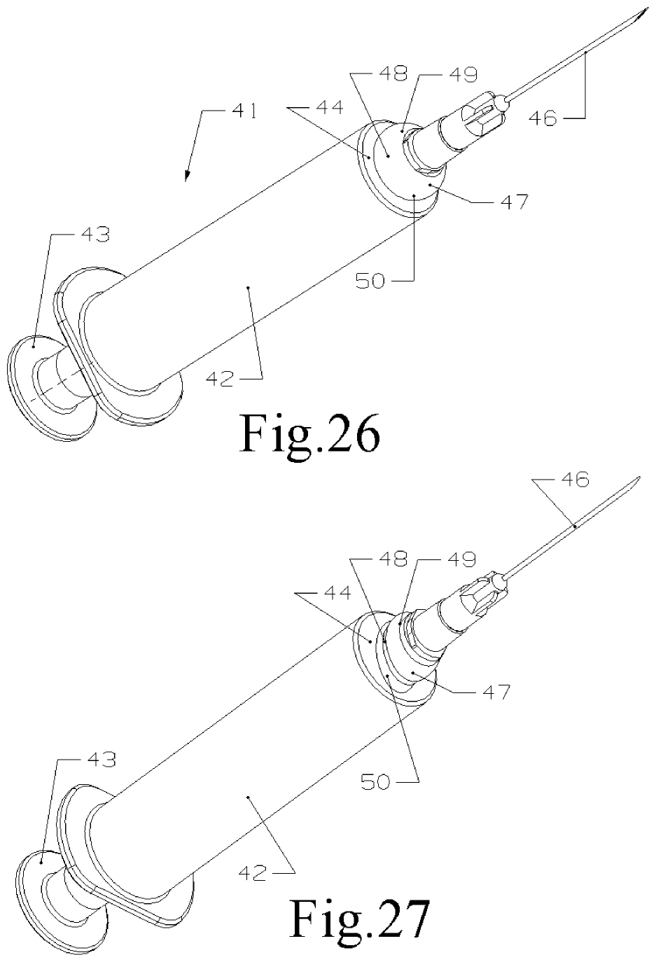

FIGS. 26-35 show a medical syringe 41 for preventing needle-stick injury according to a third embodiment of the present invention.

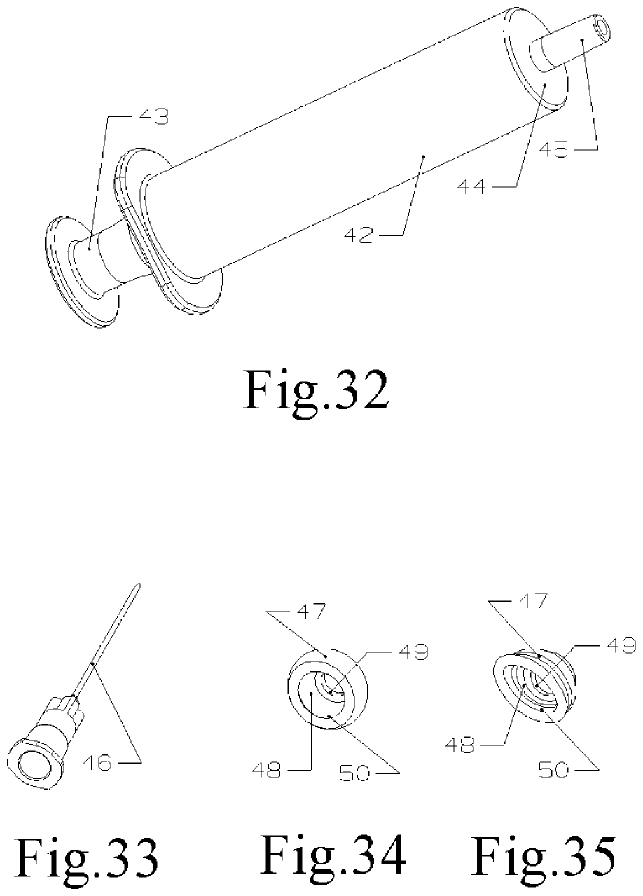

As shown in FIG. 26 and FIG. 32, the medical syringe 41 for preventing needle-stick injury includes a syringe barrel 42 and a plunger rod 43. A distal wall 44 and a conical fitting 45 that is Luer slip centric are provided at a front part of the syringe barrel 42. The distal wall 44 of the syringe barrel is a plane. The conical fitting 45 is located at a front end of the distal wall 44. After the plunger rod 43 is plugged into the syringe barrel 42 from an opening in a proximal end of the syringe barrel 42, the plunger rod 43 is capable of moving forwards and backwards in the syringe barrel 42. The medical syringe 41 for preventing needle-stick injury may be a glass medical syringe for preventing needle-stick injury or a metal medical syringe for preventing needle-stick injury.

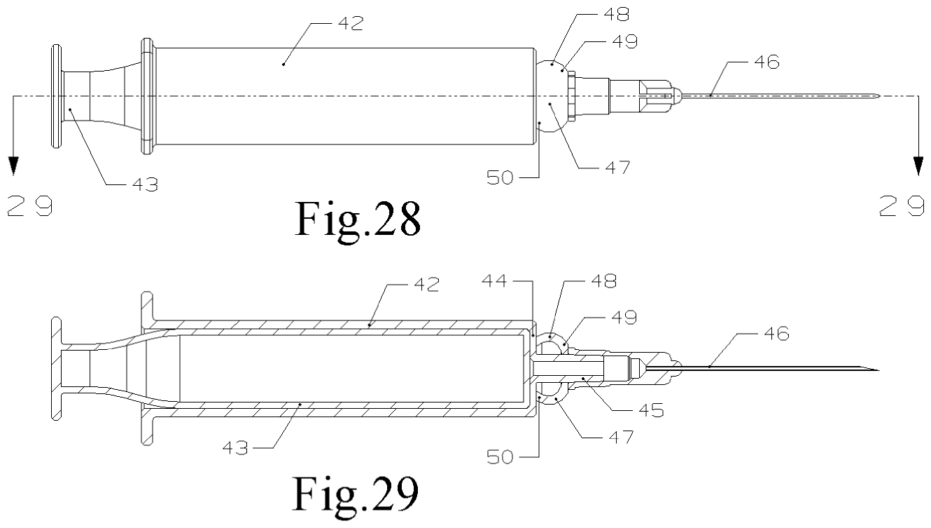

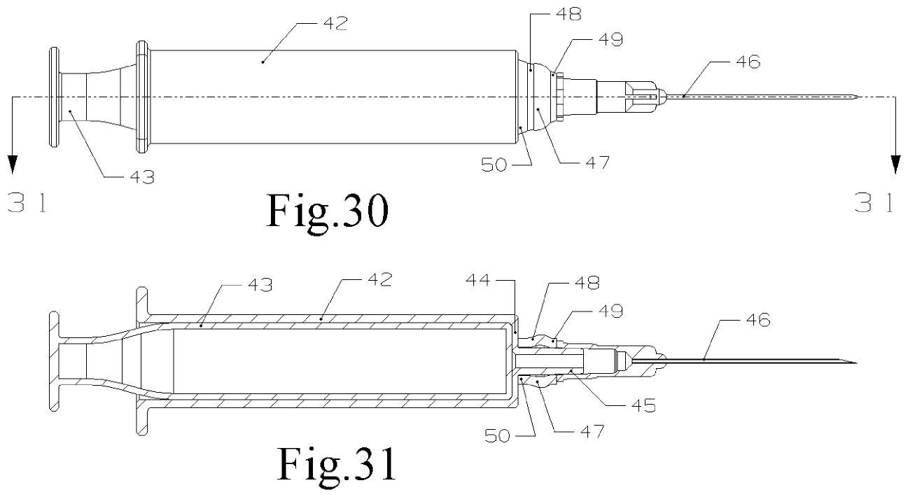

A needle 46 and an elastic disengaging ring 47 may be plugged onto the conical fitting 45 of the syringe barrel 42. The elastic disengaging ring 47 is located between the distal wall 44 of the syringe barrel 42 and the needle 46 (refer to FIG. 26). An inner diameter of a middle part 48 of the elastic disengaging ring 47 is larger than an inner diameter of a front part 49 and an inner diameter of a rear part 50 (refer to FIG. 34). The elastic disengaging ring 47 is capable of moving forwards and backwards on the conical fitting 45 of the syringe barrel 42. When the needle 46 and the elastic disengaging ring 47 are plugged onto the conical fitting 45 of the syringe barrel 42, the needle 46 is connected to the conical fitting 45 of the syringe barrel 42, and the elastic disengaging ring 47 is located between the distal wall 44 of the syringe barrel 42 and the needle 46 (refer to FIG. 26, FIG. 28 and FIG. 29). When the middle part of the elastic disengaging ring 47 on the conical fitting 45 of the syringe barrel 42 is radially pinched flat, a proximal end of the elastically deformed elastic disengaging ring 47 leans against the distal wall 44 of the syringe barrel 42, and a front end of the elastically deformed elastic disengaging ring 47 leans against a proximal end of the needle 46 and pushes the needle 46 to move forward, so that the needle 46 is disengaged from the conical fitting 45 of the syringe barrel 42 (refer to FIG. 27, FIG. 30, FIG. 31 and FIG. 35).

The inner diameter of the front part 49 of the elastic disengaging ring 47 is smaller than an outer diameter of the conical fitting 45 of the syringe barrel 42. When the inner diameter of the front part 49 of the elastic disengaging ring 47 is elastically deformed and plugged onto the conical fitting 45 of the syringe barrel 42, the elastic disengaging ring 47 is constrained on the conical fitting 45 of the syringe barrel 42, and the elastic disengaging ring 47 moves forwards and backwards on the conical fitting 45 of the syringe barrel 42 only under an external force (refer to FIG. 26, FIG. 28, FIG. 29 and FIG. 34).

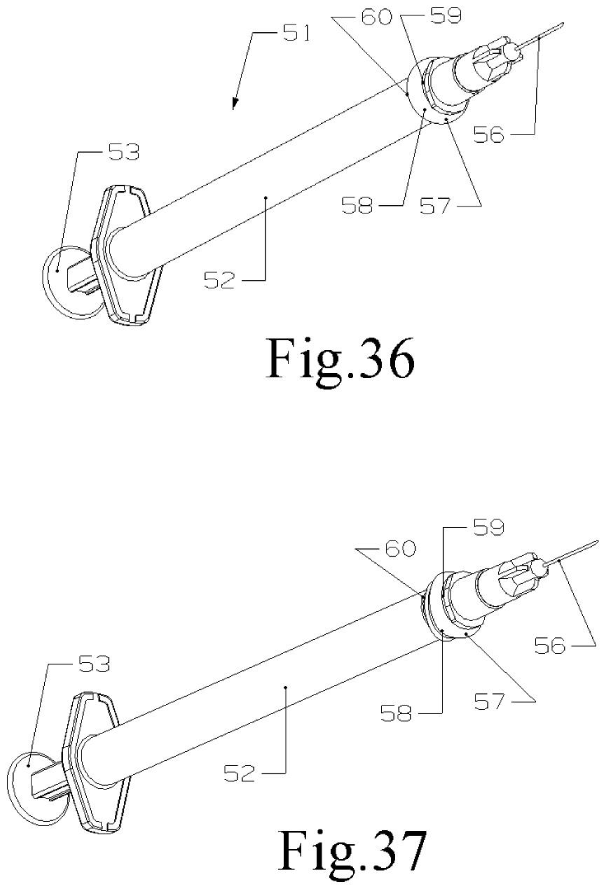

FIGS. 36-45 show a medical syringe 51 for preventing needle-stick injury according to the fourth embodiment of the present invention.

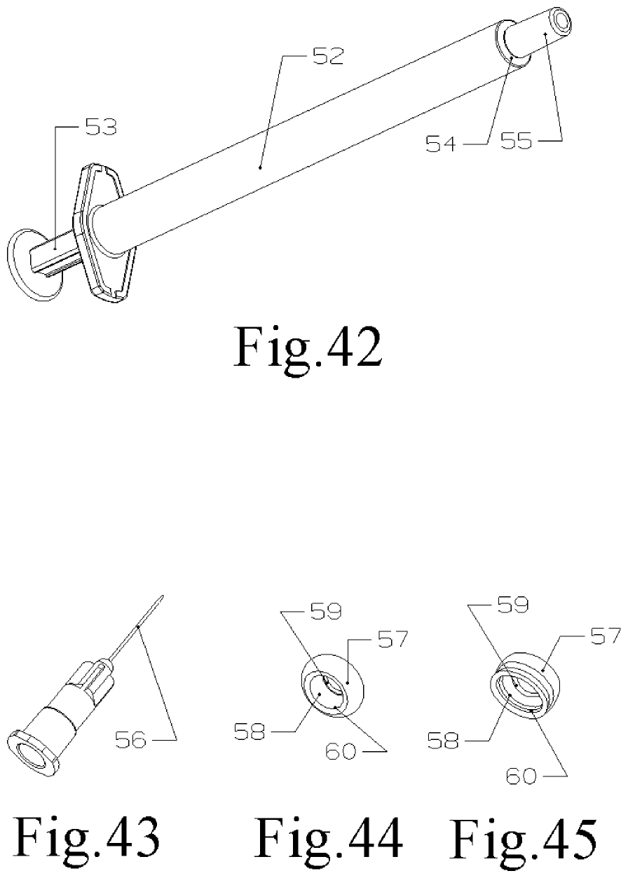

As shown in FIG. 36 and FIG. 42, the medical syringe 51 for preventing needle-stick injury includes a syringe barrel 52 and a plunger rod 53. A distal wall 54 and a conical fitting 55 that is Luer slip centric are provided at a front part of the syringe barrel 52. The conical fitting 55 is located at a front end of the distal wall 54. After the plunger rod 53 is plugged into the syringe barrel 52 from an opening in a proximal end of the syringe barrel 52, the plunger rod 53 is capable of moving forwards and backwards in the syringe barrel 52. The medical syringe 51 for preventing needle-stick injury may be a sterile syringe for single use for preventing needle-stick injury which has small volume and has a slender syringe barrel, a glass medical syringe for preventing needle-stick injury or a metal medical syringe for preventing needle-stick injury.

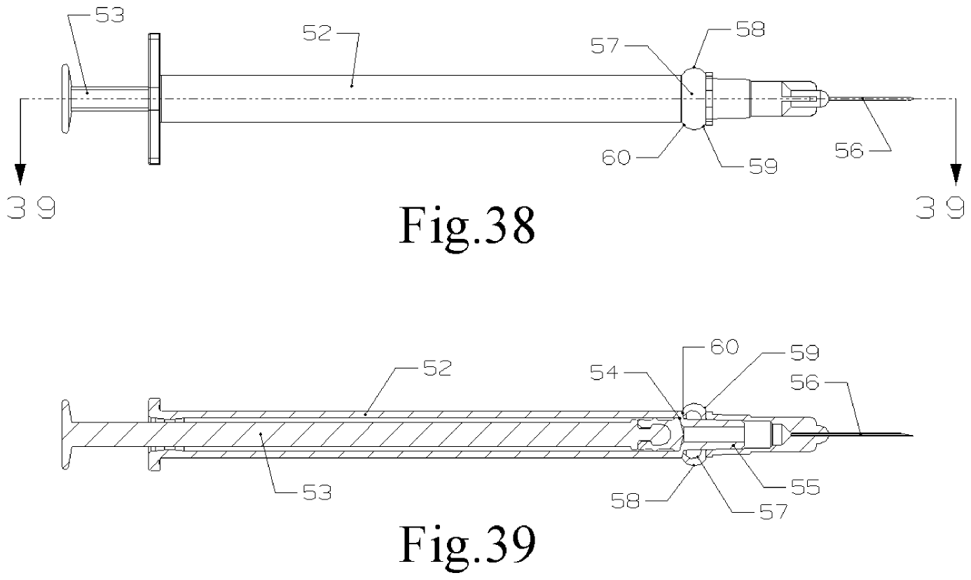

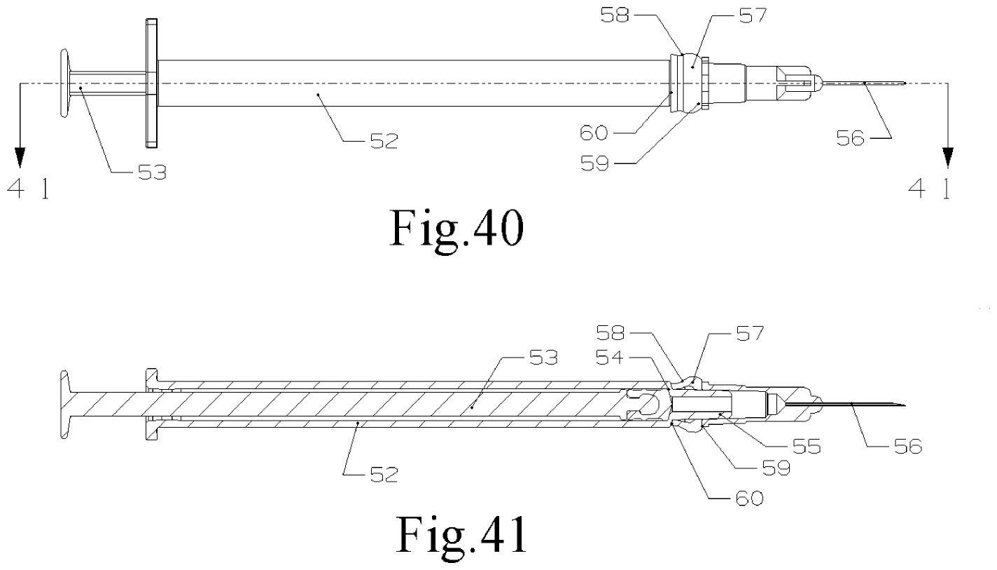

A needle 56 and an elastic disengaging ring 57 may be plugged onto the conical fitting 55 of the syringe barrel 52. The elastic disengaging ring 57 is located between the distal wall 54 of the syringe barrel 52 and the needle 56 (refer to FIG. 36). An inner diameter of a middle part 58 of the elastic disengaging ring 57 is larger than an inner diameter of a front part 59 and an inner diameter of a rear part 60 (refer to FIG. 44). The elastic disengaging ring 57 is capable of moving forwards and backwards on the conical fitting 55 of the syringe barrel 52. When the needle 56 and the elastic disengaging ring 57 are plugged onto the conical fitting 55 of the syringe barrel 52, the needle 56 is connected to the conical fitting 55 of the syringe barrel 52, and the elastic disengaging ring 57 is located between the distal wall 54 of the syringe barrel 52 and the needle 56 (refer to FIG. 36, FIG. 38 and FIG. 39). When the middle part of the elastic disengaging ring 57 on the conical fitting 55 of the syringe barrel 52 is radially pinched flat, a proximal end of the elastically deformed elastic disengaging ring 57 leans against the distal wall 54 of the syringe barrel 52, and a front end of the elastically deformed elastic disengaging ring 57 leans against a proximal end of the needle 56 and pushes the needle 56 to move forward, so that the needle 56 is disengaged from the conical fitting 55 of the syringe barrel 52 (refer to FIG. 37, FIG. 40, FIG. 41 and FIG. 45).

The inner diameter of the front part 59 of the elastic disengaging ring 57 is smaller than an outer diameter of the conical fitting 55 of the syringe barrel 52. When the inner diameter of the front part 59 of the elastic disengaging ring 57 is elastically deformed and plugged onto the conical fitting 55 of the syringe barrel 52, the elastic disengaging ring 57 is constrained on the conical fitting 55 of the syringe barrel 52, the elastic disengaging ring 57 moves forwards and backwards on the conical fitting 55 of the syringe barrel 52 only under an external force (refer to FIG. 36, FIG. 38, FIG. 39 and FIG. 44).

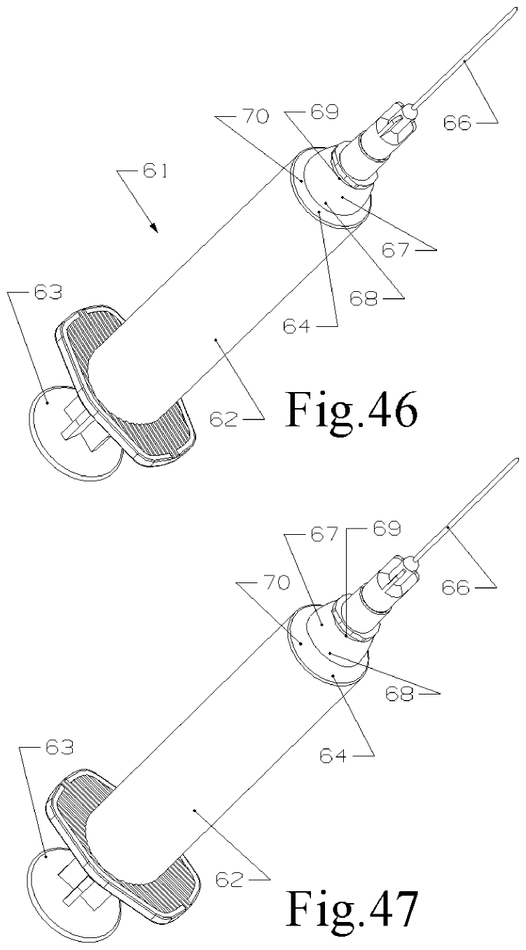

FIGS. 46-55 show a medical syringe 61 for preventing needle-stick injury according to a fifth embodiment of the present invention.

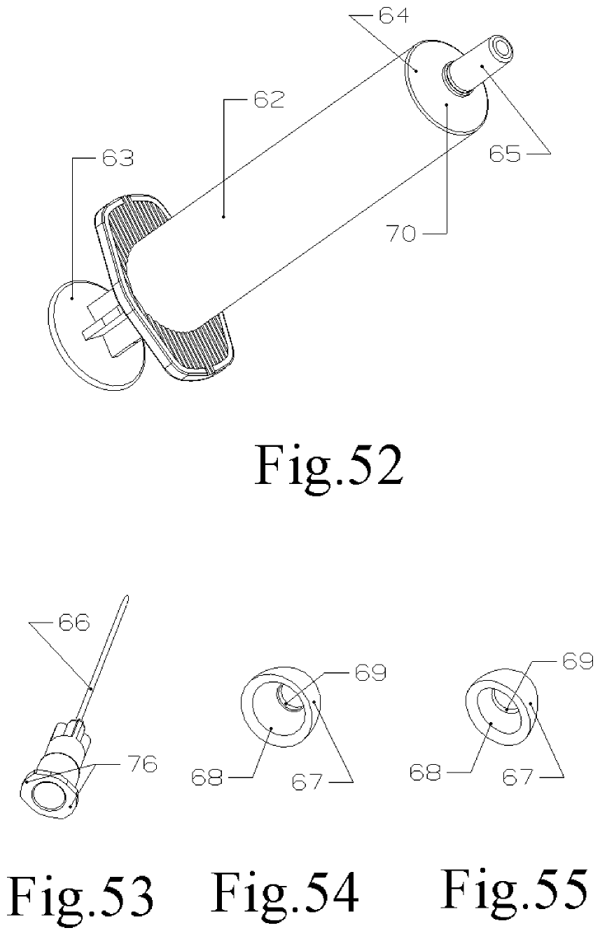

As shown in FIG. 46 and FIG. 52, the medical syringe 61 for preventing needle-stick injury includes a syringe barrel 62 and a plunger rod 63. A distal wall 64 and a conical fitting 65 that is Luer slip centric are provided at a front part of the syringe barrel 62. The conical fitting 65 is located at a front end of the distal wall 64. After the plunger rod 63 is plugged into the syringe barrel 62 from an opening in a proximal end of the syringe barrel 62, the plunger rod 63 is capable of moving forwards and backwards in the syringe barrel 62. The medical syringe 61 for preventing needle-stick injury may be a sterile syringe for single use for preventing needle-stick injury.

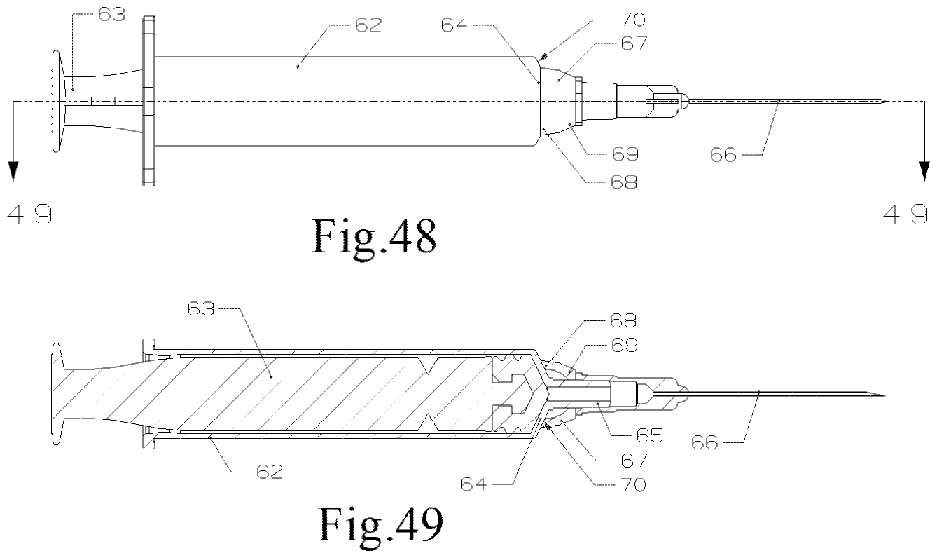

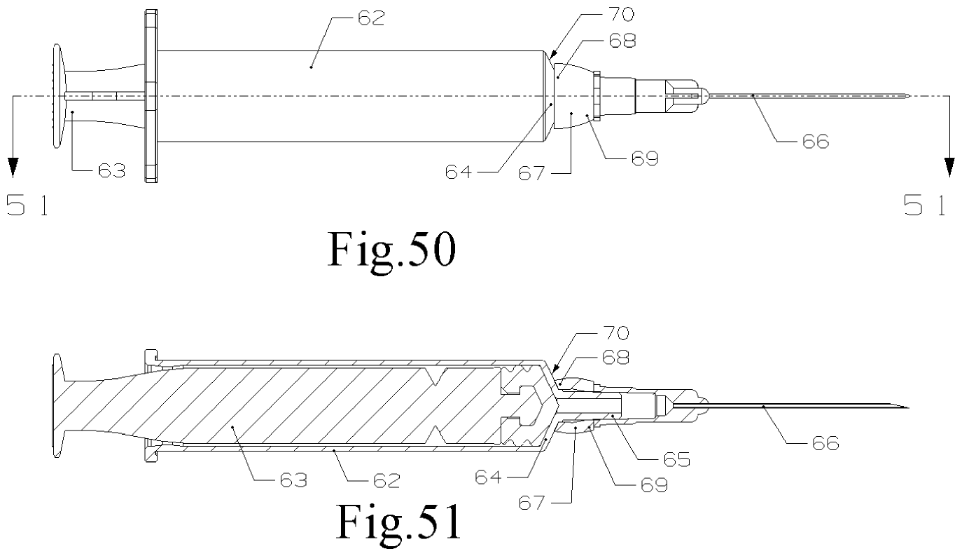

A needle 66 and an elastic disengaging ring 67 may be plugged onto the conical fitting 65 of the syringe barrel 62. The elastic disengaging ring 67 is located between the distal wall 64 of the syringe barrel 62 and the needle 66 (refer to FIG. 46, FIG. 48, FIG. 49 and FIG. 54). An inner diameter of a rear part 68 of the elastic disengaging ring 67 is larger than an inner diameter of a front part 69 of the elastic disengaging ring 67 and an outer diameter of the conical fitting 65 of the syringe barrel 62. The elastic disengaging ring 67 is capable of moving forwards and backwards on the conical fitting 65 of the syringe barrel 62. A conical inclined surface 70 is provided on the distal wall 64 of the syringe barrel 62 (refer to FIG. 52). When the rear part of the elastic disengaging ring 67 on the conical fitting 65 of the syringe barrel 62 is radially pinched flat, a proximal end of the elastically deformed elastic disengaging ring 67 leans against the distal wall 64 of the syringe barrel 62 and moves along the conical inclined surface 70 on the distal wall 64, and a front end of the elastically deformed elastic disengaging ring 67 leans against a proximal end of the needle 66 and pushes the needle 66 to move forward, so that the needle 66 is disengaged from the conical fitting 65 of the syringe barrel 62 (refer to FIG. 47, FIG. 50, FIG. 51 and FIG. 55).

The inner diameter of the front part 69 of the elastic disengaging ring 67 is smaller than an outer diameter of the conical fitting 65 of the syringe barrel 62. When the inner diameter of the front part 69 of the elastic disengaging ring 67 is elastically deformed and plugged onto the conical fitting 65 of the syringe barrel 62, the elastic disengaging ring 67 is constrained on the conical fitting 65 of the syringe barrel 62, and the elastic disengaging ring 67 moves forwards and backwards on the conical fitting 65 of the syringe barrel 62 only under an external force (refer to FIG. 46, FIG. 48, FIG. 49 and FIG. 54).

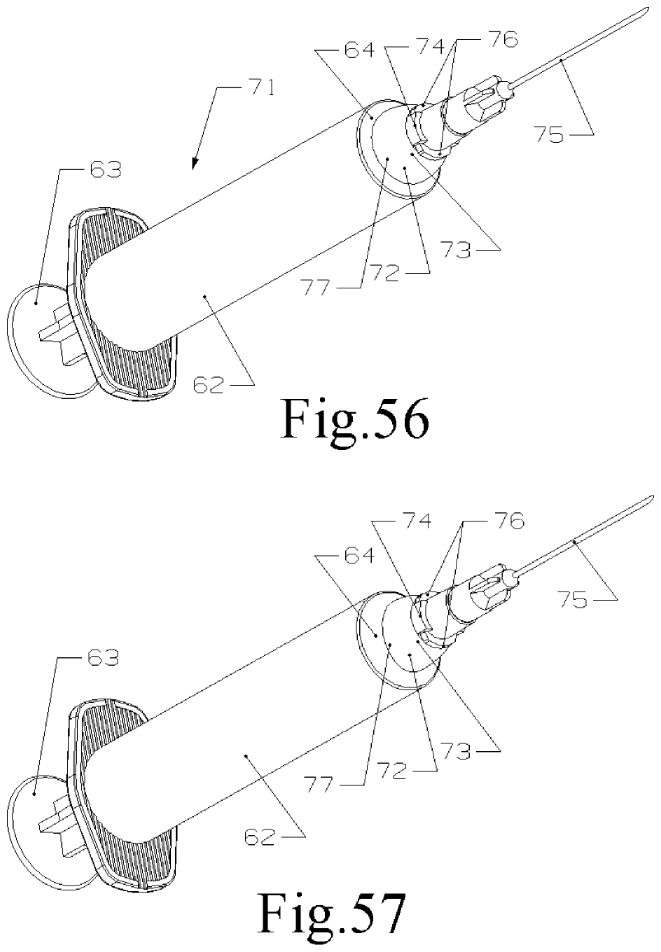

FIGS. 56-61 show a medical syringe 71 for preventing needle-stick injury according to a sixth embodiment of the present invention.

As shown in FIG. 52 and FIG. 56, the medical syringe 71 for preventing needle-stick injury includes a syringe barrel 62 and a plunger rod 63. A distal wall 64 and a conical fitting 65 that is Luer slip centric are provided at a front part of the syringe barrel 62. The conical fitting 65 is located at a front end of the distal wall 64. After the plunger rod 63 is plugged into the syringe barrel 62 from an opening in a proximal end of the syringe barrel 62, the plunger rod 63 is capable of moving forwards and backwards in the syringe barrel 62.

A needle 66 may be plugged onto the conical fitting 65 of the syringe barrel 62. As shown in FIG. 53, FIG. 56 to FIG. 61, a front part 73 of the elastic disengaging ring 72 is fixedly snap-fitted on a locking fitting 76 of a rear part of the needle 75 by a bayonet 74. An inner diameter of a rear part 77 of the elastic disengaging ring 72 is larger than an inner diameter of the front part 73 of the elastic disengaging ring 72 and an outer diameter of the conical fitting 65 of the syringe barrel 62.

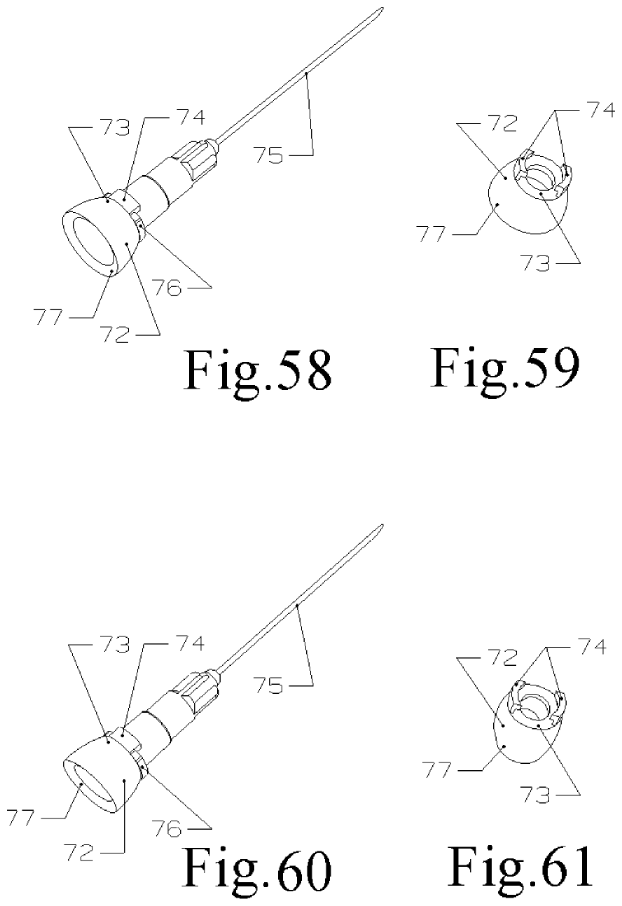

When the needle 75 is plugged onto the conical fitting 65 of the syringe barrel 62, the needle 75 is connected to the conical fitting 65 of the syringe barrel 62, and the elastic disengaging ring 72 is located between the distal wall 64 of the syringe barrel 62 and the needle 75 (refer to FIG. 56, FIG. 58 and FIG. 59). When the rear part 77 of the elastic disengaging ring 72 mounted on the rear part of the needle 75 is radially pinched flat, a proximal end of the elastically deformed elastic disengaging ring 72 leans against the distal wall 64 of the syringe barrel 62 and moves along the distal wall 64, and a front part of the elastically deformed elastic disengaging ring 72 leans against a proximal end of the needle 75 and pushes the needle 75 to move forward, so that the needle 75 is disengaged from the conical fitting 65 of the syringe barrel 62 (refer to FIG. 57, FIG. 60 and FIG. 61).

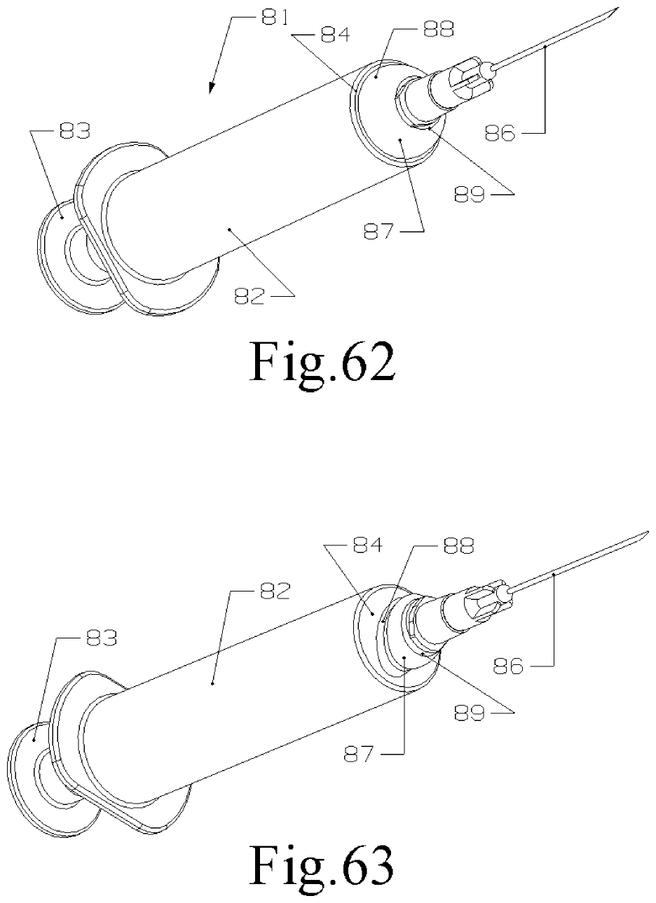

FIGS. 62-71 show a medical syringe 81 for preventing needle-stick injury according to a seventh embodiment of the present invention.

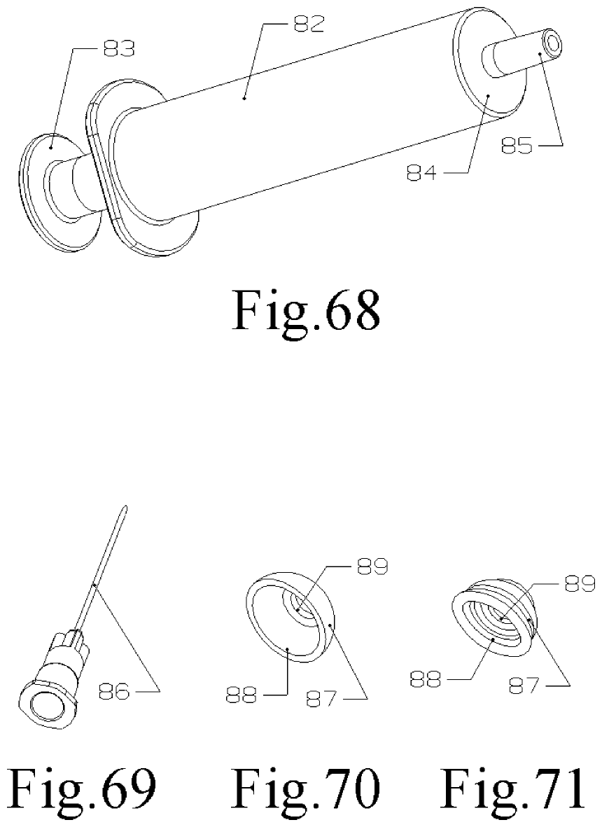

As shown in FIG. 62 and FIG. 68, the medical syringe 81 for preventing needle-stick injury includes a syringe barrel 82 and a plunger rod 83. A distal wall 84 and a conical fitting 85 that is Luer slip centric are provided at a front part of the syringe barrel 82. The distal wall 84 of the syringe barrel 82 is a plane. The conical fitting 85 is located at a front end of the distal wall 84. After the plunger rod 83 is plugged into the syringe barrel 82 from an opening in a proximal end of the syringe barrel 82, the plunger rod 83 is capable of moving forwards and backwards in the syringe barrel 82. The medical syringe 81 for preventing needle-stick injury may be a glass medical syringe for preventing needle-stick injury or a metal medical syringe for preventing needle-stick injury.

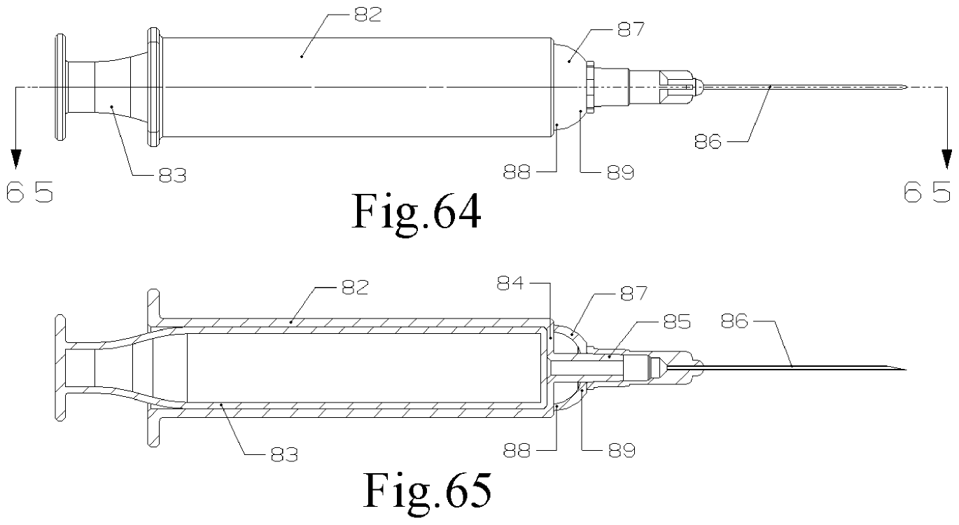

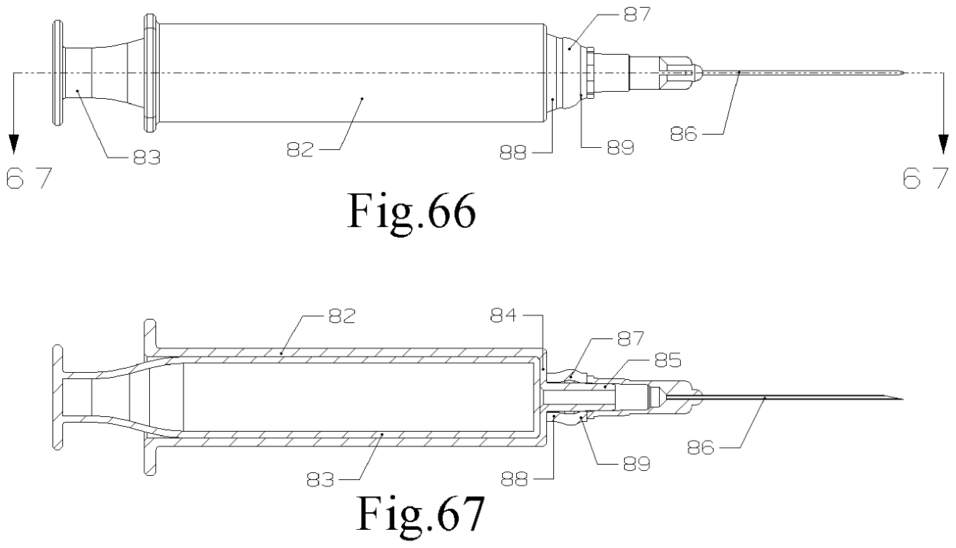

A needle 86 and an elastic disengaging ring 87 may be plugged onto the conical fitting 85 of the syringe barrel 82. The elastic disengaging ring 87 is located between the distal wall 84 of the syringe barrel 82 and the needle 86 (refer to FIG. 62). An inner diameter of a rear part 88 of the elastic disengaging ring 87 is larger than an inner diameter of a front part 89 of the elastic disengaging ring 87 and an outer diameter of the conical fitting 85 of the syringe barrel 82 (refer to FIG. 70). The elastic disengaging ring 87 is capable of moving forwards and backwards on the conical fitting 85 of the syringe barrel 82. When the needle 86 and the elastic disengaging ring 87 are plugged onto the conical fitting 85 of the syringe barrel 82, the needle 86 is connected to the conical fitting 85 of the syringe barrel 82, and the elastic disengaging ring 87 is located between the distal wall 84 of the syringe barrel 82 and the needle 86 (refer to FIG. 62, FIG. 64 and FIG. 65). When the rear part of the elastic disengaging ring 87 on the conical fitting 85 of the syringe barrel 82 is radially pinched flat, a proximal end of the elastically deformed elastic disengaging ring 87 leans against the distal wall 84 of the syringe barrel 82 and moves along the distal wall 84, and a front end of the elastically deformed elastic disengaging ring 87 leans against a proximal end of the needle 86 and pushes the needle 86 to move forward, so that the needle 86 is disengaged from the conical fitting 85 of the syringe barrel 82 (refer to FIG. 63, FIG. 66, FIG. 67 and FIG. 71).

The inner diameter of the front part 89 of the elastic disengaging ring 87 is smaller than an outer diameter of the conical fitting 85 of the syringe barrel 82. When the inner diameter of the front part 89 of the elastic disengaging ring 87 is elastically deformed and plugged onto the conical fitting 85 of the syringe barrel 82, the elastic disengaging ring 87 is constrained on the conical fitting 85 of the syringe barrel 82, and the elastic disengaging ring 87 moves forwards and backwards on the conical fitting 85 of the syringe barrel 82 only under an external force (refer to FIG. 62, FIG. 64, FIG. 65 and FIG. 70).

According to the above-mentioned detailed description, it can be seen that the medical syringe for preventing needle-stick injury provided by the present invention can achieve the following technical effects:

The medical syringe for preventing needle-stick injury according to the present invention adopts a technical feature that the inner diameter of the middle part of an elastic disengaging ring is larger than the inner diameter of the front part and the inner diameter of the rear part, and adopts a technical solution that the elastic disengaging ring is located between a distal wall of a syringe barrel and a needle. When the middle part of the elastic disengaging ring plugged onto the conical fitting of the syringe barrel is radially pinched flat, the proximal end of the elastically deformed elastic disengaging ring leans against the distal wall of the syringe barrel, and the front end of the elastically deformed elastic disengaging ring leans against the proximal end of the needle and pushes the needle to move forward, so that the needle is disengaged from the conical fitting of the syringe barrel. The technical effect that the medical syringe for preventing needle-stick injury will not touch the needle stick during operation is achieved, so as to prevent the contaminated needle from injuring the human body. Since the inner diameter of the middle part of the elastic disengaging ring is larger than the inner diameter of the front part and the inner diameter of the rear part, only a small force is required to compress the front part and the rear part of the elastic disengaging ring to each other without affecting the operation to plug the needle onto the conical fitting of the syringe barrel. When the middle part of the elastic disengaging ring is radially pinched flat, the middle part of the elastic disengaging ring is squeezed between the front part and the rear part thereof, so that the axial length of the elastic disengaging ring is elongated and the rigidity is increased, which facilitates the needle being pushed forward and then disengaging from the conical fitting of the syringe barrel. The elastic disengaging ring fitted over the conical fitting of the syringe barrel does not affect the injection operation of the medical syringe for preventing needle-stick injury, or increase the injection procedures.

The medical syringe for preventing needle-stick injury according to the present invention adopts a technical feature the inner diameter of the rear part of the elastic disengaging ring is larger than the inner diameter of the front part of the elastic disengaging ring and the outer diameter of the conical fitting of the syringe barrel, and adopts a technical solution that the elastic disengaging ring is located between the distal wall of the syringe barrel and the needle. When the rear part of the elastic disengaging ring on the conical fitting of the syringe barrel is radially pinched flat, the proximal end of the elastically deformed elastic disengaging ring leans against the distal wall of the syringe barrel and moves along the distal wall, and the front end of the elastically deformed elastic disengaging ring leans against the proximal end of the needle and pushes the needle to move forward, so that the needle is disengaged from the conical fitting of the syringe barrel. The technical effect that the medical syringe for preventing needle-stick injury will not touch the needle stick during operation is achieved, so as to prevent the contaminated needle from injuring the human body. The elastic disengaging ring fitted over the conical fitting of the syringe barrel does not affect the injection operation of the medical syringe for preventing needle-stick injury, or increase the injection procedures.

The present invention provides a medical syringe for preventing needle-stick injury, which has only one more elastic disengaging ring over the conical fitting of the syringe barrel than the conventional medical syringe. The cost of the elastic disengaging ring is less than 3% of the cost of the medical syringe for preventing needle-stick injury, and the assembly is also convenient. The elastic disengaging ring can be used to form a sterile syringe for single use for preventing needle-stick injury by directly putting one elastic disengaging ring on the conical fitting of the syringe barrel of conventional various sterile syringes for single use. For sterile syringe for single use for preventing needle-stick injury in the present invention, needle can be replaced with needles of other specifications before use; after use, the contaminated metal needles and plastic medical syringes can be classified into a collection box, the plastic material and the metal material can be classified and recycled, or subsequently destroyed. Conventional various glass medical syringes and metal medical syringes can be routinely cleaned and sterilized before use. When in use, one elastic disengaging ring can be put on the conical fitting of the syringe barrel to form a glass medical syringe for preventing needle-stick injury or a metal medical syringe for preventing needle-stick injury, the elastic disengaging ring and the needle are destroyed after use. Since the medical syringe for preventing needle-stick injury of the present invention can directly use conventional syringe barrels, plunger rods and needles of conventional various sterile syringes for single use, various glass medical syringes or various metal medical syringes, therefore existing processing, assembly and packaging techniques and equipment can be directly applied, thereby reducing the product cost. The conventional needles which can be used both for various medical syringes with an outer conical locking fitting and for various medical syringes without the outer conical locking fitting will not be affected.

Therefore, it can be seen that the objectives, including the objectives shown by the above descriptions, are effectively achieved. Only typical and preferred embodiments of the present invention are described herein, and some changes may be made to the aforementioned structures without departing from the spirit and scope of the present invention. The present invention is not limited to or confined by the described specific details, and shall include, as stated in the claims, any improvement or modification obvious to persons of ordinary skill in the art.

What is claimed is:

1. A medical syringe for preventing needle-stick injury, comprising:
a syringe barrel, wherein a distal wall and a conical fitting are provided at a front part of said syringe barrel in which said conical fitting is located at a front end of said distal wall;
a plunger rod being capable of moving forwards and backwards in the syringe barrel after said plunger rod is inserted into the syringe barrel from an opening of a proximal end of the syringe barrel; and
a needle and an elastic disengaging ring, wherein said needle and said elastic disengaging ring are plugged onto the conical fitting of the syringe barrel and the elastic disengaging ring is capable of moving forwards and backwards on the conical fitting of the syringe barrel, and wherein the elastic disengaging ring is located between the distal wall of the syringe barrel and the needle when the needle and the elastic disengaging ring are plugged onto the conical fitting of the syringe barrel, and at this time, wherein a proximal end of the elastically deformed elastic disengaging ring leans against the distal wall of the syringe barrel and a front end of the elastically deformed elastic disengaging ring leans against a proximal end of the needle and pushes the needle to move forward so that the needle is disengaged from the conical fitting of the syringe barrel when the elastic disengaging ring plugged onto the conical fitting of the syringe barrel is radially pinched flat.

2. A medical syringe for preventing needle-stick injury according to claim 1, wherein an inner diameter of a middle part of said elastic disengaging ring is larger than an inner diameter of a front part and an inner diameter of a rear part, and wherein the elastic disengaging ring is located between said distal wall of said syringe barrel and said needle when the needle and the elastic disengaging ring are plugged onto said conical fitting of the syringe barrel, and at this time, wherein the proximal end of the elastically deformed elastic disengaging ring leans against the distal wall of the syringe barrel and the front end of the elastically deformed elastic disengaging ring leans against the proximal end of the needle and pushes the needle to move forward so that the needle is disengaged from the conical fitting of the syringe barrel when the middle part of the elastic disengaging ring plugged onto the conical fitting of the syringe barrel is radially pinched flat.

3. A medical syringe for preventing needle-stick injury according to claim 1, wherein an inner diameter of a rear part of said elastic disengaging ring is larger than an inner diameter of a front part of the elastic disengaging ring and an outer diameter of said conical fitting of said syringe barrel, and wherein the elastic disengaging ring is located between said distal wall of the syringe barrel and said needle when the needle and the elastic disengaging ring are plugged onto the conical fitting of the syringe barrel, and at this time, wherein the proximal end of the elastically deformed elastic disengaging ring leans against said distal wall of the syringe barrel and moves along the distal wall and the front end of the elastically deformed elastic disengaging ring leans against the proximal end of the needle and pushes the needle to move forward so that the needle is disengaged from the conical fitting of the syringe barrel when the rear part of the elastic disengaging ring plugged onto the conical fitting of the syringe barrel is radially pinched flat.

4. A medical syringe for preventing needle-stick injury according to claim 1, wherein an inner diameter of a rear part of said elastic disengaging ring is larger than an outer diameter of said conical fitting of said syringe barrel and a conical inclined surface is provided on said distal wall of the syringe barrel, and wherein the elastic disengaging ring is located between the distal wall of the syringe barrel said the needle when the needle and the elastic disengaging ring are plugged onto the conical fitting of the syringe barrel, and at this time, wherein the proximal end of the elastically deformed elastic disengaging ring leans against the distal wall of the syringe barrel and moves along said conical inclined surface on the distal wall and the front end of the elastically deformed elastic disengaging ring leans against the proximal end of the needle and pushes the needle to move forward so that the needle is disengaged from the conical fitting of the syringe barrel when the elastic disengaging ring on the conical fitting of the syringe barrel is radially pinched flat.

5. A medical syringe for preventing needle-stick injury according to claim 1, wherein an inner diameter of a front part of said elastic disengaging ring is smaller than an outer diameter of said conical fitting of said syringe barrel, and wherein the elastic disengaging ring is constrained on the conical fitting of the syringe barrel and the elastic disengaging ring is capable of moving forwards and backwards on the conical fitting of the syringe barrel only under an external force when the inner diameter of the front part of the elastic disengaging ring is elastically deformed and plugged onto the conical fitting of the syringe barrel.

6. A medical syringe for preventing needle-stick injury, comprising:
a syringe barrel, wherein a distal wall and a conical fitting are provided at a front part of said syringe barrel in which said conical fitting is located at a front end of said distal wall;
a plunger rod being capable of moving forwards and backwards in the syringe barrel after said plunger rod is inserted into the syringe barrel from an opening of a proximal end of the syringe barrel; and
a needle and an elastic disengaging ring, wherein said needle and said elastic disengaging ring are plugged onto the conical fitting of the syringe barrel and a front part of the elastic disengaging ring is fixedly snap-fitted on a locking fitting of a rear part of the needle by a bayonet, and wherein the elastic disengaging ring is located between the distal wall of the syringe barrel and the needle when the needle and the elastic disengaging ring are plugged onto the conical fitting of the syringe barrel, and at this time, wherein a proximal end of the elastically deformed elastic disengaging ring leans against the distal wall of the syringe barrel and a front end of the elastically deformed elastic disengaging ring leans against a proximal end of the needle and pushes the needle to move forward so that the needle is disengaged from the conical fitting of the syringe barrel when the elastic disengaging ring plugged onto the conical fitting of the syringe barrel is radially pinched flat.

7. A medical syringe for preventing needle-stick injury according to claim 6, wherein an inner diameter of a middle part of said elastic disengaging ring is larger than an inner diameter of a front part and an inner diameter of a rear part, and wherein the elastic disengaging ring is located between said distal wall of said syringe barrel and said needle when the needle and the elastic disengaging ring are plugged onto said conical fitting of the syringe barrel, and at this time, wherein the proximal end of the elastically deformed elastic disengaging ring leans against the distal wall of the syringe barrel and the front end of the elastically deformed elastic disengaging ring leans against the proximal end of the needle and pushes the needle to move forward so that the needle is disengaged from the conical fitting of the syringe barrel when the middle part of the elastic disengaging ring plugged onto the conical fitting of the syringe barrel is radially pinched flat.

8. A medical syringe for preventing needle-stick injury according to claim 6, wherein an inner diameter of a rear part of said elastic disengaging ring is larger than an inner diameter of a front part of the elastic disengaging ring and an outer diameter of said conical fitting of said syringe barrel, and wherein the elastic disengaging ring is located between said distal wall of the syringe barrel and said needle when the needle and the elastic disengaging ring are plugged onto the conical fitting of the syringe barrel, and at this time, wherein the proximal end of the elastically deformed elastic disengaging ring leans against the distal wall of the syringe barrel and moves along the distal wall and the front end of the elastically deformed elastic disengaging ring leans against the proximal end of the needle and pushes the needle to move forward so that the needle is disengaged from the conical fitting of the syringe barrel when the rear part of the elastic disengaging ring plugged onto the conical fitting of the syringe barrel is radially pinched flat.

9. A medical syringe for preventing needle-stick injury according to claim 6, wherein an inner diameter of a rear part of said elastic disengaging ring is larger than an outer diameter of said conical fitting of said syringe barrel and a conical inclined surface is provided on said distal wall of the syringe barrel, and wherein the elastic disengaging ring is located between the distal wall of the syringe barrel and said needle when the needle and the elastic disengaging ring are plugged onto the conical fitting of the syringe barrel, and at this time, wherein the proximal end of the elastically deformed elastic disengaging ring leans against the distal wall of the syringe barrel and moves along said conical inclined surface on the distal wall and the front end of the elastically deformed elastic disengaging ring leans against the proximal end of the needle and pushes the needle to move forward, so that the needle is disengaged from the conical fitting of the syringe barrel when the elastic disengaging ring on the conical fitting of the syringe barrel is radially pinched flat.

10. A medical syringe for preventing needle-stick injury, comprising:
a syringe barrel, wherein a distal wall and a conical fitting are provided at a front part of said syringe barrel in which said conical fitting is located at a front end of said distal wall;
a plunger rod being capable of moving forwards and backwards in the syringe barrel after said plunger rod is inserted into the syringe barrel from an opening of a proximal end of the syringe barrel; and
a needle and an elastic disengaging ring, wherein said elastic disengaging ring is fixedly mounted at a front part of the distal wall of the syringe barrel, and wherein the elastic disengaging ring is located between the distal wall of the syringe barrel and said needle when the needle is plugged onto the conical fitting of the syringe barrel, and at this time, wherein a proximal end of the elastically deformed elastic disengaging ring leans against the distal wall of the syringe barrel and a front end of the elastically deformed elastic disengaging ring leans against a proximal end of the needle and pushes the needle to move forward so that the needle is disengaged from the conical fitting of the syringe barrel when the elastic disengaging ring plugged onto the conical fitting of the syringe barrel is radially pinched flat.

11. A medical syringe for preventing needle-stick injury according to claim 10, wherein an inner diameter of a middle part of said elastic disengaging ring is larger than an inner diameter of a front part and an inner diameter of a rear part, and wherein the elastic disengaging ring is located between said distal wall of said syringe barrel and said needle when the needle is plugged onto the conical fitting of the syringe barrel, and at this time, wherein the proximal end of the elastically deformed elastic disengaging ring leans against the distal wall of the syringe barrel and the front end of the elastically deformed elastic disengaging ring leans against the proximal end of the needle and pushes the needle to move forward so that the needle is disengaged from the conical fitting of the syringe barrel when the middle part of the elastic disengaging ring plugged onto the conical fitting of the syringe barrel is radially pinched flat.
